(19) United States
(12) Patent Application Publication
(10) Pub. No.: US 2025/0152677 A1
(43) Pub. Date: May 15, 2025

(54) TREATMENT OF LIVER CANCERS BY DISRUPTING THE BETA-CATENIN/TCF-4 BINDING SITE LOCATED UPSTREATM OF MEG3 IN THE DLK1/DIO3 LOCUS

(71) Applicants: INSTITUT NATIONAL DE LA SANTÉ ET DE LA RECHERCHE MÉDICALE, Paris (FR); UNIVERSITÉ PARIS CITÉ, Paris (FR); SORBONNE UNIVERSITÉ, Paris (FR); CENTRE NATIONAL DE LA RECHERCHE SCIENTIFIQUE, Paris (FR); INOVARION, Paris (FR)

(72) Inventors: Angélique GOUGELET, Paris (FR); Julie SANCEAU, Paris (FR); Lucie POUPEL, Paris (FR); Sabine COLNOT, Paris (FR)

(21) Appl. No.: 18/835,868

(22) PCT Filed: Feb. 13, 2023
(86) PCT No.: PCT/EP2023/053419

§ 371 Date: Aug. 5, 2024

(30) Foreign Application Priority Data
Feb. 14, 2022 (EP) 22305162.4

(51) Int. Cl. A61K 38/46; A61P 35/00; C12N 15/11; C12N 15/86

(52) U.S. Cl. CPC A61K 38/465; A61P 35/00; C12N 15/11; C12N 15/86; C12N 2310/20; C12N 2750/14143

(57) ABSTRACT

Activating mutations in CTNNB1 gene encoding β-catenin is encountered in approximately 30% of hepatocellular carcinoma (HCC) and in more than 80% of hepatoblastoma. In Apc^{Δhep} model, the inventors unravel the biggest cluster of non-coding RNAs identified called the DLK1/DIO3 locus as the most significantly induced region in response to β-catenin activation regarding transcription of coding and non-coding elements. Using in vivo Crispr/cas9 strategy, the inventors were able to demonstrate that β-catenin and its cofactor TCF-4 directly bind on a WRE-containing site located upstream of Meg3 to create an active enhancer regulatory region engaged in chromatin remodeling in the direct vicinity of this binding site but also at distance by long range DNA-DNA contacts to promote transcription of the entire locus. These Crispr/cas9 constructs have also proved to be a valuable strategy to impair the locus expression in the murine models mimicking HCC and hepatoblastoma (Apc^{Δhep} and β-catenin^{ΔExon3} tumors) but also in two cell lines with activating mutations in β-catenin encoding gene, the murine Hepa1-6 and human HuH6 cells. In transformed cells, it significantly impaired cell proliferation in vitro and HuH6 sternness capacities but also tumor progression in Hepa1-6 allografts. In mouse models, the locus editing during early steps of tumorigenesis decreased the proliferation of Apc^{Δhep} preneoplastic hepatocytes but also those of Apc^{Δhep} and β-catenin^{ΔExon3} tumor cell resulting in impairment of tumor size. In conclusion, the results demonstrate that disrupting the β-catenin/TCF-4 binding site located upstream of Meg3 in the DLK1/DIO3 locus represents a very interesting approach for the treatment of liver cancers.

### FIELD OF THE INVENTION

The present invention is in the field of medicine, in particular oncology.

### BACKGROUND OF THE INVENTION

Discovered in the late 1980s by two scientific groups¹, the β-catenin plays a plethora of functions during embryonic development and adult homeostasis but also in disease, functioning as a transcriptional co-regulator in the canonical Wnt/β-catenin pathway². This molecule transduces Wnt-activating signals potentiated by R-spondin at the cell membrane³ into the nucleus to modify the transcription of a cell-specific repertoire of target genes. However, the β-catenin cannot bind directly at the promoter regions of its target genes and needs to interact with TCF/Lef (T-cell factor/lymphoid enhancer factor) transcription factors as β-catenin binding partners on specific WRE (Wnt responsive elements) sites⁴. Besides its intricate link with TCF/Lef, the β-catenin is able to build multi-protein complexes in the vicinity of its targets to establish an epigenetic and chromatin context in favour or against transcription⁵. The transcriptional switch orchestrated by the β-catenin is notably related to modulation of chromatin structure either by recruiting histone acetyltransferases such as CBP, p300, or Tip60, or by rearranging nucleosome position notably driven by SWI/SNF ((SWItch/Sucrose Non-Fermentable) and ISWI (Imitation SWI)⁶. β-catenin/CBP complexes are thus associated with modifications in H₃K₂₇ acetylation, as it has been shown on myogenic genes during differentiation into myotubes, while it modifies acetylation driven by KAT2B on H₃K₉ residues on cell cycle and proliferation genes in myoblasts⁷. In addition, β-catenin is able to recruit members of the COMPASS complex such as MLL1 (mixed-lineage-leukemia), leading to H₃K₄ trimethylation, a mark associated with transcribed region⁸. Finally, the β-catenin also interferes with long-range DNA-DNA interactions to create DNA loops with higher local concentration of the required factors and associated with transcription activation or repression, i.e., in Myc promoter region in colon cancer cells⁹.

Close relationships between the Wnt/β-catenin pathway and cell proliferation dates back from Wnt1a discovery forty years ago and a broad spectrum of evidence has shown that β-catenin interferes with cell cycle and mitosis at different level¹⁰. Firstly, several components of the Wnt signaling pathways are recognized as important actor in microtubule dynamics during mitosis¹¹. Importantly, the levels of β-catenin and of its regulated gene repertoire are modified during cell cycle and rise to a peak at the G2/M phase¹². Secondly, cyclin D1, a key player in the G1/S transition, is one β-catenin direct target¹³, and particularly engaged in liver regeneration during partial hepatectomy¹⁴. Finally, during oncogenesis, β-catenin also plays a crucial role for the emergence and expansion of progenitors located in the vicinity of the Herring canal¹⁵ and its aberrant activation leads to an increased liver to body weight ratio in part due to hepatocyte proliferation¹⁶.

Regarding the myriad of key roles played by the β-catenin in cell fate and determination, any dysfunction causes developmental defects or diseases and notably cancers. Mutations in Wnt/β-catenin signaling are frequently observed in cancers such as pulmonary, colorectal or liver cancers. In particular, activating mutations in its encoding gene CTNNB1 is encountered in around 10% of cancers and detected in around 30% of hepatocellular carcinoma (HCC), the most frequent primitive liver cancer¹⁷ but also arise in more than 80% of hepatoblastoma, a pediatric liver tumor¹⁸. In adult, HCCs arise in a context of chronic disease, in which hepatocytes die and adjacent hepatocytes leave their quiescent state to proliferate and compensate this loss of integrity. These cycles of necrosis and proliferation are prone to the emergence of new oncogenic mutations accelerating tumor initiation¹⁹. If numerous studies have been conducted to unveil co-occurrent mutations in liver cancers, the kinetic of these pro-oncogenic events from dysplastic nodules to advanced tumors remains nowadays largely unknown.

These latter years, our laboratory has expanded its efforts to decipher how the β-catenin could impact hepatocyte fate through its transcriptional regulatory roles on metabolic, proliferative and epigenetic actors using a transgenic mouse model of aberrant activation of this pathway (Apc^{Δhep})²⁰⁻²³. As previously mentioned in ²⁰, as the top induced target in Apc^{Δhep}, we identified a large region of 800 kb located in chromosome 12 in mouse and called the DLK1/DIO3 locus. The DLK1/DIO3 locus is an imprinted locus containing the largest clusters of non-coding RNAs: 54 miRNAs including miR-127 and miR-136 but also several snoRNAs and the three long non-coding RNAs Meg3, Mirg and Rian. Paternally-derived RNAs are only expressed in a healthy adult liver and its imprinting and expression is mostly regulated by methylation of its DMR regions (differentially methylated regions) divided into three sites called DLK1-, IG- and MEG3-DMR with different regulatory functions²⁴. The miR-379/miR-410 cluster is central for metabolic adaptation after weaning²⁵, while elevated Meg3 is associated with gluconeogenesis, hepatic insulin resistance and cholestasis²⁶. In cancer, studies mainly focus on individual RNAs deriving from the locus, and often found these RNAs downregulated in close association with a bad prognosis²⁷. However, in non-small cell lung cancer, adrenocortical carcinoma and in hepatoblastoma, the expression of the DLK1/DIO3 locus is associated with a bad prognosis²⁸⁻³⁰. Interestingly, in adrenocortical carcinoma as well as in hepatoblastoma, induction of the DLK1/DIO3 locus is associated with activating mutations in the Wnt/β-catenin pathway.

### SUMMARY OF THE INVENTION

The present invention is defined by the claims. In particular, the present invention relates to the treatment of a liver cancer by disrupting the β-catenin/TCF-4 binding site located upstream of Meg3 in the DLK1/DIO3 locus.

### DETAILED DESCRIPTION OF THE INVENTION

Activating mutations in CTNNB1 gene encoding β-catenin is encountered in around 10% of cancers and arise in approximately 30% of hepatocellular carcinoma (HCC) and in more than 80% of hepatoblastoma. These subgroups of tumors present peculiar epigenetic and immune features with impact on their prognosis. Using dedicated mouse models, inventors' works conducted this decade aim at understanding how an oncogenic activation of this pathway impacts the preneoplastic hepatocyte transcriptome, epigenome and metabolome in favor of tumor emergence. In Apc^{Δhep} model, the inventors unravel the biggest cluster of non-coding RNAs identified called the DLK1/DIO3 locus as the most significantly induced region in response to β-catenin activation regarding transcription of coding and non-coding elements. Using in vivo Crispr/cas9 strategy, the inventors were able to demonstrate that β-catenin and its cofactor TCF-4 directly bind on a WRE-containing site located upstream of Meg3 to create an active enhancer regulatory region engaged in chromatin remodeling in the direct vicinity of this binding site but also at distance by long range DNA-DNA contacts to promote transcription of the entire locus. These Crispr/cas9 constructs have also proved to be a valuable strategy to impair the locus expression in the murine models mimicking HCC and hepatoblastoma (Apc^{Δhep} and β-catenin^{ΔExon3} tumors) but also in two cell lines with activating mutations in β-catenin encoding gene, the murine Hepa1-6 and human HuH6 cells. In transformed cells, it significantly impaired cell proliferation in vitro and HuH6 stemness capacities but also tumor progression in Hepa1-6 allografts. In mouse models, the locus editing during early steps of tumorigenesis decreased the proliferation of Apc^{Δhep} preneoplastic hepatocytes but also those of Apc^{Δhep} and β-catenin^{ΔExon3} tumor cell resulting in impairment of tumor size. Deep-sequencing analyses conducted on DLK1/DIO3^{ΔWRE} hepatocytes showed that the DLK1/DIO3 locus impacts actors involved in cytokinesis and cell cycle progression and notably mitosis entry. The inventors finally showed by ChIP experiments that the β-catenin-driven deregulation of cell cycle involving the locus is partly due to FoxM1 redistribution on the promoter regions of its main targets such as cyclin A2 and Kif20a. In conclusion, the present work identified the DLK1/DIO3 locus as a key oncogenic event subsequent to β-catenin mutations through reprogramming of proliferation gene signatures. It explains for the first time why this locus is induced in certain types of tumors and how the β-catenin directly drives this reprogramming through an enhancer regulatory region upstream of Meg3, which could constitute an interesting therapeutic target specifically for this subgroup of tumors. More importantly, the results demonstrate that disrupting the β-catenin/TCF-4 binding site located upstream of Meg3 in the DLK1/DIO3 locus represents a very interesting approach for the treatment of liver cancers.

### Main Definitions

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein the term “wild type” is a term of the art understood by skilled persons and means the typical form of an organism, strain, gene or characteristic as it occurs in nature as distinguished from mutant or variant forms.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “liver cancer” refers to liver carcinomas notably hepatocellular carcinoma (HCC)) as well as other tumors of liver (e.g., cholangiocarcinoma (bile duct cancers), combined hepatocellular carcinoma and cholangiocarcinoma, and hepatoblastoma). Liver cancers generally develop in patients with risk factors that include alcohol abuse, viral hepatitis, and metabolic liver disease.

As used herein, the term “hepatocellular carcinoma” or “HCC” has its general meaning in the art and refers to a malignant tumor of hepatocellular origin. HCC can undergo hemorrhage and necrosis because of a lack of fibrous stroma. Vascular invasion, particularly of the portal system, is common. Aggressive HCC can cause hepatic rupture and hemoperitoneum.

As used herein, the term “hepatoblastoma” has its general meaning in the art and refers to a pediatric liver cancer that arises from precursors of hepatocytes and can have several morphologies, including the following:
Small cells that reflect neither epithelial nor stromal differentiation. It is critical to discriminate between small cell undifferentiated hepatoblastoma expressing SMARCB1 and rhabdoid tumor of the liver, which lacks the SMARCB1 gene and SMARCB1 expression. Both diseases may share similar histology. Optimal treatment of rhabdoid tumor of the liver and small cell undifferentiated hepatoblastoma may require different approaches and different chemotherapy. (Refer to the Small cell undifferentiated histology hepatoblastoma and rhabdoid tumors of the liver section of this summary for a more extensive discussion of the differences between small cell undifferentiated hepatoblastoma and rhabdoid tumor of the liver.)
Embryonal epithelial cells resembling the liver epithelium at 6 to 8 weeks of gestation.
Well-differentiated fetal hepatocytes morphologically indistinguishable from normal fetal liver cells.

Most often the tumor consists of a mixture of epithelial hepatocyte precursors. About 20% of tumors have stromal derivatives such as osteoid, chondroid, and rhabdoid elements. Occasionally, neuronal, melanocytic, squamous, and enteroendocrine elements are found.

As used herein, the term “β-catenin” has its general meaning in the art and refers to the protein that in humans is encoded by the CTNNB1 gene (Kraus C, Liehr T, Hülsken J, Behrens J, Birchmeier W, Grzeschik K H, Ballhausen W G (September 1994). “Localization of the human beta-catenin gene (CTNNB1) to 3p21: a region implicated in tumor development”. Genomics. 23 (1): 272-4). The term is also known as catenin beta-1. β-catenin is a dual function protein, involved in regulation and coordination of cell-cell adhesion and gene transcription. β-catenin is a subunit of the cadherin protein complex and acts as an intracellular signal transducer in the Wnt signaling pathway. Mutations and overexpression of β-catenin are associated with many cancers, including hepatocellular carcinoma, colorectal carcinoma, lung cancer, malignant breast tumors, ovarian and endometrial cancer (Morin P J (December 1999). “beta-catenin signaling and cancer”. BioEssays. 21 (12): 1021-30).

As used herein, the expression “activating mutation in the CTNNB1 gene” has its general meaning in the art and refers to any mutation in the CTNNB1 gene that leas to the constitutive activity of β-catenin. Most of these mutations cluster on a tiny area of the N-terminal segment of β-catenin i.e. the B-TrCP binding motif and thus make ubiquitinylation and degradation of β-catenin impossible. It will cause β-catenin to translocate to the nucleus without any external stimulus and continuously drive transcription of its target genes. The activating mutations in the CTNNB1 gene are well known in the art and are typically described in Forbes S A, Bindal N, Bamford S, Cole C, Kok C Y, Beare D, Jia M, Shepherd R, Leung K, Menzies A, Teague J W, Campbell P J, Stratton M R, Futreal P A (January 2011). “COSMIC: mining complete cancer genomes in the Catalogue of Somatic Mutations in Cancer”. Nucleic Acids Research. 39 (Database issue): D945-50. These mutations produce elevated levels of Tcf4-beta-catenin complexes, which stimulates a transcriptional response that promotes malignant growth.

As used herein, the term “TCF4” has its general meaning the art and refers to the transcription factor 4 that is a basic helix-loop-helix transcription factor. TCF4 is a member of the TCF/Lef family transcription factors. TCF4 interact with β-catenin to form the “TCF4-β-catenin complex” that stimulates a transcriptional response that promotes malignant growth. Accordingly, the term “TCF4-β-catenin binding site” refers to a site present on DNA whereby the TCF4-β-catenin complex binds. The TCF4-β-catenin binding site is a WRE-containing site, i.e. a site that comprises at least one WRE motif (e.g. the consensus sequence is TCAAAG) (SEQ ID NO:1) but some substitutions are authorized in certain positions).

As used herein, the term “DLK1/DIO3 locus” has its general meaning in the art and refers to a large region of 800 kb located in chromosome 14 in humans. The DLK1/DIO3 locus is an imprinted locus containing the largest clusters of non-coding RNAs: 54 miRNAs including miR-127 and miR-136 but also several snoRNAs and the three long non-coding RNAs Meg3, Mirg and Rian (Gougelet, A. et al. Antitumour activity of an inhibitor of miR-34a in liver cancer with beta-catenin-mutations. Gut 65, 1024-1034, doi: 10.1136/gutjnl-2014-308969 (2016)).

As used herein, the term “Meg3” has its general meaning in the art and refers to a long noncoding RNA encoded by the maternally expressed gene 3 (MEG3) gene (Al-Rugeebah A, Alanazi M, Parine N R. MEG3: an Oncogenic Long Non-coding RNA in Different Cancers. Pathol Oncol Res. 2019 July;25 (3): 859-874. doi: 10.1007/s12253-019-00614-3. Epub 2019 Feb. 21. PMID: 30793226).

As used herein, the term “β-catenin/TCF-4 binding site located upstream of MEG3” refers to the genomic sequence as set forth in SEQ ID NO:2.

>sequence of the catenin/TCF4 binding site
located upstream of Meg3
SEQ ID NO: 2
ATTGCTTGAGCCCAGGAGTTTGAGGCTGCAATGAACCATGATTGCACCAC
TGTACTCCAGCCTGGGCAACAAAGTGAGATCTCGTCCCAAAAAAAGATAA
ATAAAAAGAAAATAGTCTGTTTTAGTCTGTATTCAGAGCAAGCCTGTGGC
ATGAATATCAACTTTCCTGTTTTTGCAGGGGGGAAACCGAGGCCTGGCAG
GGCGAAGTGGGCAGGACCCTTCTCAAAGGGCCAGGGTGCTAGTGGCCACT
GTCCTTCCCCCACCACCTCCACGCCCCCATCCCCCCACAGTTCACGACTG
CAAAGGCAGCTTTAGGTTGGGAGCTGGTGGAGCAAAAAGGCCGTTCAAAG
GCAGCCTTCGTTTGCTTTTCCTTATCATCACATGTGCAAGGGCAGCTCCG
TTTACCTAGAGGCAGGCGTCTTTGAGCTCATCCTTTCCGCAAACACCTAA
AGGGTATGTTGAACATTTGAGCCCCTGTCTCAGAGGGCGCAGCGTTTGCA
CAACATAAGCCCTGGCGGCTCGGTAGACGGGTATGGGGGGTAGACTCAGG
TTTCTGGATGAAGCACTGAGAGCCAAGGTGACCTGCGGTGTTCCGGGATG
ACCTTTGGTGACCTTCGGTCCTGTCCAGTGACCTGGGGTGGTGAAGTTAC
CCCAAATGGGAGGAAATGAACTCTGGGTGGGCGAAGGCTGCAGGACGTGT
TGGCATGATGGCTGTAGAAAGTGCACACCAGGGCTGGTGTGGAGGGCGCC
TCGGCTGCTCTGAGAATCTTCCACTACCCCGGCCTGGGATTCCCAGGAAA
GGCTGGTGGTGGAGGCCAGAGAGGCTCA

As used herein, the term “agent capable of disrupting the β-catenin/TCF-4 binding site” refers to any agent that is capable of allowing the genome editing the binding site of the β-catenin/TCF-4 complex thereby preventing the binding of said transcriptional complex to said site.

As used herein, the term “DNA targeting endonuclease” has its general meaning in the art and refers to an endonuclease that generates a double-strand break (DSB) at a desired position in the genome without producing undesired toxic off-target DSBs. The DNA targeting endonuclease can be a naturally occurring endonuclease (e.g., a bacterial meganuclease) or it can be artificially generated (e.g., engineered meganucleases, TALENs, or ZFNs, among others).

As used herein the term “cleaves” generally refers to the generation of a double-strand break in the DNA genome at a desired location. Cleavage thus results in alteration of the genome sequence by non-homologous end joining (NHEJ) repair system or microhomology mediated end joining (MMEJ) repair system. According to the present invention alteration by NHEJ repair system is preferred.

As used herein, the term “genome editing” of the genomic sequence includes a replacement of one or more nucleotides, the insertion of one or more nucleotides, and/or the deletion of one or more nucleotides anywhere within a genome.

As used herein, the term “TALEN” has its general meaning in the art and refers to a transcription activator-like effector nuclease, an artificial nuclease which can be used to edit a target gene. TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29:149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29:135-6; and Boch et al. (2009) Science 326:1509-12; Moscou et al. (2009) Science 326:3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29:149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29:143-8; Hockemeyer et al. (2011) Nature Biotech. 29:731-734; Wood et al. (2011) Science 333:307; Doyon et al. (2010) Nature Methods 8:74-79; Szczepek et al. (2007) Nature Biotech. 25:786-793; and Guo et al. (2010) J. Mol. Biol. 200:96). The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29:143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17:11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

As used herein, the term “Zinc Finger Nuclease” or “ZFN” has its general meaning in the art and refers to a zinc finger nuclease, an artificial nuclease which can be used to edit a target gene. Like a TALEN, a ZFN comprises a DNA-modifying domain, e.g., a nuclease domain, e.g., a FokI nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188:773-782; and Kim et al. (1996) Proc. Natl. Acad. Sci. USA 93:1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95:10570-5). Also like a TALEN, a ZFN can create a DSB in the DNA, which can create a frame-shift mutation if improperly repaired, e.g., via non-homologous end joining, leading to a decrease in the expression of a target gene in a cell.

As used herein, the term “CRISPR-associated endonuclease” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

As used herein, the term “Cpf1 protein” to a Cpf1 wild-type protein derived from Type V CRISPR-Cpf1 systems, modifications of Cpf1 proteins, variants of Cpf1 proteins, Cpf1 orthologs, and combinations thereof. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532 (7600): 517-21 (2016)).

As used herein, the term “guide RNA” or “gRNA” has its general meaning in the art and refers to an RNA which can be specific for a target DNA and can form a complex with the CRISPR-associated endonuclease. A guide RNA can comprise a spacer sequence that specifies a target site and guides an RNA/Cas complex to a specified target DNA for cleavage. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. The specific sequence of the guide RNA may vary, but, regardless of the sequence, useful guide RNA sequences will be those that minimize off-target effects while achieving high efficiency of alteration at the targeted loci. The length of the spacer sequence can vary from about 17 to about 60 or more nucleotides, for example about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein.

As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “viral vector” refers to a virion or virus particle that functions as a nucleic acid delivery vehicle and which comprises a vector genome packaged within the virion or virus particle. Typically, the vector is a viral vector which is an adeno-associated virus (AAV), a retroviral vector, bovine papilloma virus, an adenovirus vector, a vaccinia virus, or a polyoma virus.

As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences.

As used herein, the term “treatment” or “treat” refers to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day.

Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

### METHODS OF THE PRESENT INVENTION

The first object of the present invention relates to a method of treating a liver cancer in a patient harbouring at least one activating mutation in CTNNB1 gene comprising administering to the patient an agent capable of disrupting the β-catenin/TCF-4 binding site located upstream of Meg3 in the DLK1/DIO3 locus.

In particular, the method of the present invention comprises administering to the patient a therapeutically effective amount of a DNA-targeting endonuclease whereby the DNA-targeting endonuclease cleaves the genomic DNA of the cancer cells in at least one position present in the β-catenin/TCF-4 binding site and thereby repressing the transcription of the DLK1/DIO3 locus.

In some embodiments, the DNA-targeting endonuclease of the present invention leads to the genome editing of the β-catenin/TCF-4 binding site located upstream of Meg3 so that the transcriptional β-catenin/TCF-4 complex is not able to bind to its binding site.

In some embodiments, the patient suffers from a hepatocellular carcinoma.

In some embodiments, the patient suffers from a hepatoblastoma.

In some embodiments, the DNA targeting endonuclease of the present invention is a TALEN. In some embodiments, the DNA targeting endonuclease of the present invention is a ZFN. In some embodiments, the DNA targeting endonuclease of the present invention is a CRISPR-associated endonuclease.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomona aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, MA).

In some embodiments, the CRISPR-associated endonuclease is a Cpf1 nuclease.

In some embodiments, nucleotide sequence encoding for the nuclease (e.g. Cas9 or Cpf1) can be modified to encode biologically active variants of said nuclease, and these variants can have or can include, for example, an amino acid sequence that differs from a wild type nuclease by virtue of containing one or more mutations (e.g., an addition, deletion, or substitution mutation or a combination of such mutations). One or more of the substitution mutations can be a substitution (e.g., a conservative amino acid substitution). For example, a biologically active variant of a nuclease polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type nuclease polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The nuclease sequence can be a mutated sequence. For example, the Cas9 nuclease can be mutated in the conserved FiNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks.

In some embodiments, the method of the present invention comprises administering an effective amount of a CRISPR-associated endonuclease with one or more guide RNA.

In some embodiments, the guide RNA is used for recruiting the CRISPR-associated endonuclease to the β-catenin/TCF-4 binding site located upstream of Meg3 and generating DSBs.

The guide RNA molecule of the present invention thus comprises a guide sequence for providing the targeting specificity. It includes a region that is complementary and capable of hybridization to a pre-selected target site of interest.

In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed: PAM sequence in the relevant genome. The user may be allowed to choose the length of the seed sequence. The user may also be allowed to specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

In some embodiments, the guide RNA targets a sequence selected from SEQ ID NO:3-7. In some embodiments, the guide RNA targets the sequence SEQ ID NO:5 or SEQ ID NO:6. In some embodiments, a plurality of guide RNAs is used. In some embodiments, a combination of 2 guide RNAs is used wherein the first guide RNA targets the sequence SEQ ID NO:4 and the second guide RNA targets at least one sequence selected from the group consisting of SEQ ID NO: 3, SEQ ID NO:5 and SEQ ID NO:6.

>Sq3
SEQ ID NO: 3
GCAGGACCCTTCTCAAAGGGC (Sg3)
>Sg2Bis
SEQ ID NO: 4
TCCGCAAACACCTAAAGGGTA (Sg2Bis)
>Sg5
SEQ ID NO: 5
CCACAGTTCACGACTGCAAAG (Sg5)
>Sg1
SEQ ID NO: 6
TATTCAGAGCAAGCCTGTGGC (Sg1)

In some embodiments, the guide RNA is encoded by the sequence selected from SEQ ID NO: 7-10. In some embodiments, a combination of 2 guide RNAs is used wherein the first guide RNA is encoded by SEQ ID NO:8 and the second guide RNA is encoded by a sequence selected from the group consisting of SEQ ID NO:7, SEQ ID NO:9, and SEQ ID NO: 10.

>Sg3
SEQ ID NO: 7
GCAGGACCCTTCTCAAAGGGCCAGGGT (Sg3)
>Sg2Bis
SEQ ID NO: 8
TACCCTTTAGGTGTTTGCGGAAAGGAT (Sg2Bis)
>Sg5
SEQ ID NO: 9
CTTTGCAGTCGTGAACTGTGGGGGGAT (Sg5)
>Sg1
SEQ ID NO: 10
TATTCAGAGCAAGCCTGTGGCATGAAT (Sg1)

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, the CRISPR-associated endonuclease and the guide RNA are provided through expression from one or more vectors. In some embodiments, the CRISPR endonuclease can be encoded by the same nucleic acid as for the guide RNA sequences. Alternatively or in addition, the CRISPR endonuclease can be encoded in a physically separate nucleic acid from the guide RNA sequences or in a separate vector. Suitable vector backbones include, for example, those routinely used in the art such as plasmids, viruses, artificial chromosomes, BACs, YACs, or PACs. Suitable vectors include derivatives of SV40 and known bacterial plasmids, e.g., E. coli plasmids col El, pCRI, pBR322, pMal-C2, pET, pGEX, pMB9 and their derivatives, plasmids such as RP4; phage DNAs, e.g., the numerous derivatives of phage 1, e.g., NM989, and other phage DNA, e.g., MI 3 and filamentous single stranded phage DNA. Vectors also include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Typically, the vector is a viral vector, and more particularly an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector and more particularly an AAV8 vector and even more particularly a AAV2/AAV8 vector.

Typically, the nucleic acid molecule or the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”. In some embodiments, the promoter is specifically select for driving the expression of the nucleic acid molecule specifically in hepatocytes.

In some embodiments, the the CRISPR-associated endonuclease and the guide RNA are provided through the use of an RNA-encoded system. In particular, the CRISPR-associated endonuclease is provided through the use of a chemically modified mRNA together with modified guide RNA as described in Jiang, T., Henderson, J. M., Coote, K. et al. Chemical modifications of adenine base editor mRNA and guide RNA expand its application scope. Nat Commun 11, 1979 (2020). In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

The invention will be further illustrated by the following figures and examples. However, these examples and figures should not be interpreted in any way as limiting the scope of the present invention.

### FIGURES

FIG. 1: Crispr/Cas9 editing of the DLK1/DIO3 locus impairs the protumorigenic capacities of cancer cell lines mutated for β-catenin

A-B: Measurement of cell proliferation by Xcelligence on Hepa1-6 (A) or HuH6 (B) edited

(DLK1/DIO3^{ΔWRE}) or not for the locus. Results are represented as the mean proliferation of all experiments normalized to control cells at 48h (N=6). C: Measurement of stemness capacities of Huh6 cells by spheroid formation assay. Results are represented as the number of spheroids divided into 4 subgroups according to their sizes. All experiments were conducted in triplicates. D-F: allograft experiments conducted on Nu/Nu mice with Hepa1-6 cells. D: Results represent tumor progression with the mean tumor sizes measured during 18 days (N=6). E: Ki-67 detection in tumors by immunohistochemistry. Results represent the percentage of tumor cells with labelled nuclei. F: Cleaved-caspase 3 detection in tumors by immunohistochemistry. Results represent the percentage of tumor cells with labelled cytosols. P<0,05: *, p<0,01: **, p<0,005: ***, p<0.001: ****, ns: non-significant. G-H: xenograft experiments conducted on Nu/Nu mice with HUH-6 cells. Results represent tumor progression with the mean tumor sizes measured during 18 days (N=12) (G) and tumor mass at sacrifice (H, left panel). Right panel: Ki-67 detection in tumors by immunohistochemistry. Results represent the percentage of tumor cells with labelled nuclei.

FIG. 2. A: Tumor incidence of undifferentiated Apc^{Δhep} tumors related to hepatoblastoma either edited (DLK1/DIO3^{ΔWRE}) or not (Rosa26). Some tumors are not edited after DLK1/DIO3^{ΔWRE} construct injection and called non-DLK1/DIO3^{ΔWRE}. B: Tumor incidence of differentiated β-catenin^{ΔExon3} tumors related to HCC either edited (DLK1/DIO3^{ΔWRE}) or not (Rosa26).

### EXAMPLE

### Material and Methods

### In Vivo CRISPR Design and Editing Analysis.

Small guides RNAs (sgRNAs) were designed using CRISPR RGEN online tool (Cas-Designer http://www.rgenome.net/cas-designer/). Twenty-one nucleotide long guides were selected according to their GC content (20% to 80%) and no potential off-target sites. Guides were subcloned in pX601 (Addgene #61591) and pX602 (Addgene #61593), single vector AAV8-Cas9 system containing Cas9 from Staphylococcus aureus driven by CMV (cytomegalovirus) promoter or TBG (thyroxine binding globulin) promoter respectively²⁷. Plasmids were then transfected into murine Hepa1-6 cells and human Huh6 cells. Two days after transfection, DNA was extracted and amplified by PCR to measure editing efficiency. For mouse experiments, recombinant AAV8 with pX602 plasmids were produced by the Center of viral vector production (Health Research Institute, Université de Nantes). For in vivo editing of the DLK1-WRE site in mice, the following sgRNAs were used: sg2 located upstream the DLK1-WRE site: TTCCTCAGTGGGGCTAAAGGAGAGGGT and sg5 located downstream: GGATGACCTTTGACTTCTGAAGGGAGT. A sgRNA against Rosa26 locus was used as a control and injected at the same dose: CTCGATGGAAAATACTCCGAGGCGGAT. In human cell lines, we used four combinations of sgRNAs to edit one or both WREs sg1:

TATTCAGAGCAAGCCTGTGGCATGAAT,
sg2:
TACCCTTTAGGTGTTTGCGGAAAGGAT,
sg3:
GCAGGACCCTTCTCAAAGGGCCAGGGT,
sg5:
CTTTGCAGTCGTGAACTGTGGGGGGAT.

### Gene Editing Analysis.

Total livers, primary hepatocytes or cultured clones were lysed overnight at 56° C. in 50 mM Tris-HCl pH8, 50 mM EDTA, 100 mM NaCl, 1% SDS buffer supplemented with proteinase K at 0.8 mg/ml. DNA extractions were performed using classical phenol-chloroform protocol Ultrapure Phenol: Chloroform: Isoamyl Alcohol (25:24:1, v/v) (Thermofisher). 400 ng of purified DNA were amplified for 40 cycles with Taq HS (Ozyme) according to manufacturer's protocol. The PCR product for Rosa26 locus were sequenced by Sanger method (Eurofins) and editing efficiency was quantified by using TIDE (Tracking of Indels by DEcomposition; https://tide.deskgen.com/). For the DLK1-WRE site, PCR products were run on E-Gel 2% for 10 minutes (Thermofischer) and bands were quantified with ImageJ. For mouse cells, the PCR primers used were as follows: DLK1-WRE F: AGCATGGCCGAGTACTCATT, DLK1-WRE R: CCTCTGCATGACCTGTGACT, ROSA26 F: CTTGCTCTCCCAAAGTCGCT, ROSA26 R: CCAATGCTCTGTCTAGGGGT. For human clones, DLK1-WRE F: TGAGGCTGCAATGAACCATG, DLK1-WRE R: GGCTTATGTTGTGCAAACGC

### Murine Models

As previously published, accordingly to the percentage of hepatocytes with Apc deletion (Apc^{Δhep}), we could either study preneoplastic or tumor phases^{18,20,28}. The Apc^{Δhep} model could be sequentially associated with CRISPR/Cas9 editing strategy against the region of interest, here the DLK1-WRE site. Retro-orbital injections of AAV8 particles were done in 2-month-old male mice under isoflurane anesthesia as follows: 3.6×10¹¹ Vg sg2 plus 1.9×10¹¹ Vg sg5 (DLK1/DIO3^{ΔWRE}). Apc deletion was then conducted one month after, which is the time that we have identified as optimal for gene editing²¹. For Apc^{lox/lox} TTR-Cre mice receiving 2 mg tamoxifen (MP Biomedicals), a deletion of Apc was observed in approximately 90% of hepatocytes at sacrifice six days after injection and allow to study the early steps of liver carcinogenesis (pretumoral Apc^{Δhep} model). To obtain tumors within 4 to 6 months, a unique injection of 109Vg Ad5-Cre-GFP was performed resulting in the deletion of Apc in 10 to 50% Apc^{lox/lox} hepatocytes, as previously described²⁸. Tumor apparition was followed monthly by 2D-ultrasound (Vevo 770) as previously published¹⁸.

Recently, we have also created a mouse model deleting exon 3 of Ctnnb1 with CRISPR/Cas9 editing²¹ (β-catenin^{ΔExon3}). To avoid immune reaction against two successive injections of AAV8, all sgRNAs against the DLK1-WRE site and the exon 3 of Ctnnb1 are concomitantly administered to 2-month-old male mice under isoflurane anesthesia and tumor apparition followed by 2D-ultrasound as described above.

For hepatocyte sorting, retro-orbital injections of 1.5x109 particles of an Ad5-Cre-GFP adenovirus were performed on two-month-old Apc^{lox/lox} mice and GFP-positive hepatocytes were sorted with an ARIA3 (BD). GFP-negative cells or GFP+ cells from Apc^{wt/wt} mice were used as control cells with no activation of β-catenin.

All animal procedures were carried out according to French legal regulations and approved by an ethical committee (agreements 2877 and 17-082).

### Human Samples

For the hepatoblastoma cohort from Bordeaux, total RNA was extracted from 48 frozen tumoral and adjacent non-tumor tissues surgically resected from 24 patients using the mirVana kit (Thermo Fisher Scientific) according to the supplier's protocol. The RNA integrity number was evaluated by Agilent 2100 Bioanalyzer. Two μg were used for generation of each small RNA library with an Illumina TruSeq®Small RNA Sample Prep Kit according to standard protocol. Single read 50 nt sequencing was performed by MGX-Montpellier GenomiX platform on an Illumina HiSeq 2000 using the Sequence By Synthesis technique. Adapter sequences were trimmed from small RNA reads using the Cutadapt (version 1.4.1) tool [http://code.google.com/p/cutadapt/], retaining reads of the size 16-25 nt. Reads were then mapped to the human hairpin sequences (mirBase version 21) with Bowtie (v1.0). The number of reads mapping in the sense orientation to each hairpin in each patient was used as an input for further analysis. Data were analyzed using DESeq2 package in R studio. The miRNA expression file was loaded in format .txt to obtain a matrix with the value in i-th row corresponding to miRNA names and value in j-th column corresponding to patient samples. We followed the DESeq2 manual⁵¹, performed differential miRNA expression analysis (DE) and produced output files including heatmaps and tables. After the DE analysis DESeq2 produces a set of values: base mean, log 2 fold change and adjusted pvalue for each miRNA between tumoral and non-tumor samples. Once exported, our data were classified in an excel file and analyzed with the help of miRBase and UCSC Browser [https://genome.ucsc.edu/]. For the liver cancer cohort from Paris, RNA sequencing was performed in both adult (N=225) and pediatric (N=123) liver samples as previously described^{3,30,31}. Gene expression levels were calculated using the variance stabilizing transformation (VERSUST) and the raw count matrix. Gene expression-based classification of HB and HCC was done as previously described^{3,52}. RT-qPCR validation for miRNA and RNA expression was conducted on 44 patients treated for liver cancer at Cochin hospital (see online supplementary table S3¹⁸). Statistical analysis and data visualization were performed using R software version 3.6.1 (R Foundation for Statistical Computing, Vienna, Austria, https://www.R-project.org) and Bioconductor packages.

### Cell Culture

For primary culture, hepatocytes were isolated by a standard collagenase perfusion protocol¹ four days after tamoxifen injection in Apc^{lox/lox} TTR-Cre mice. Cells were dispersed in William's medium (supplemented with 10% fetal bovine serum, 50U/mL penicillin-streptomycin, 0.5 μg/mL amphotericin B, 25 nM dexamethasone, 4 μg/mL insulin, and 1% bovine serum albumin), and plated on dishes coated with rat-tail collagen I (Thermofischer). Hepa1-6 cells were obtained from the American Type Culture Collection and Huh6 from C. Perret's lab². Cell lines were maintained in DMEM medium supplemented with 10% fetal calf serum and 50U/mL Penicillin/streptomycin and incubated at 37° C. in a humidified atmosphere containing 5% CO₂. Stable DLK1/DIO3^{ΔWRE} or control clones were obtained following co-transfections of plasmids containing sgRNAs with a pmax-GFP plasmid, GFP+ and GFP-negative cell sorting using ARIA3 cytometer and a unique sorted cell was cultured and amplified in 96-well plate. Each amplified clone was then selected on the basis of efficient editing.

### Proliferation/Cell Cycle Analysis

Cell adhesion and proliferation were measured with the xCELLigence system (Agilent). 8,000 cells were seeded on Xcelligence plate and the impedance was read every 5 minutes for 4 hours then every 15 minutes for 48h. All experiments were conducted at least in triplicates. For cell cycle analysis, clones were synchronized for 24 h by fetal calf serum deprivation for Huh6 and treatment with 10 μg/mL colchicine for Hepa1-6, which are more refractory to serum deprivation. 24h later, cells were rinsed and fresh medium was added for 24h. 5×10⁵cells were rinsed in PBS and fixed in PBS/ethanol (20/80) at −20° C. for 15 min. Fixed cells were then suspended in FxCycle PI/RNAse staining solution (ThermoFischer Scientific) for 30 min at room temperature before analysis on a Fortessa cytometer at 488 nm (BD).

### Sphere Formation Assay

One thousand Huh6 clones were plated onto ultra-low attachment 6-well plates (Corning) and cultured in DMEM/F12 medium with B27 supplement, 20 ng/ml EGF, 20 ng/mL basic fibroblast growth factor and 100 μg/mL gentamycin (Thermofischer Scientific) during 14 days. All experiments were conducted in triplicates.

### Allografts/Xenografts

Mice were maintained in accordance with the French guidelines for the care of laboratory animals (agreement No 14009). Subcutaneous allografts with 2E6 Hepa1-6 cells were performed on both flanks in 5-weeks old female Nu/Nu nude mice as reported elsewhere³. All experiments were conducted on at least 5 mice for each condition. Subcutaneous xenografts with 2E6 HuH-6 cells were performed on both flanks in 5-weeks old female Nu/Nu nude mice as reported elsewhere³. All experiments were conducted on at least 11 mice for each condition.

### RNA Extraction and RT-qPCR

Total RNA was extracted from hepatocytes, liver tumors, or livers with Trizol reagent according to manufacturer protocol (Thermofischer). Levels of miRNAs were determined on 10 ng total mRNA, with a specific Taqman miRNA assay and normalized to snoRNA135, which we have previously identified as the most stable non-coding RNA in our models4. Levels of mRNA were determined on 100 ng total mRNA relative to 18S RNA. The primers used were as follows: 18S F: GTA-ACC-CGT-TGA-ACC-CCA-TT, 18S R: CCA-TCC-AAT-CGG-TAG-TAG-CG, Mirg F: GCC-ATC-TAC-CTC-TGA-GTC-CC, Mirg R: AGA-GCA-GAA-ACC-CCT-CCT-TC, Rian F: CCA-GGT-TCA-AGG-TCC-CTC-AT, Rian R: TCT-TGT-GTC-TCG-AAG-GCC-TT, mki67 F: CTG-CCT-GCG-AAG-AGA-GCA-TC, mki67 R: AGC-TCC-ACT-TCG-CCT-TTT-GG, Kif20a F: AAG-TGG-TGA-GCG-GCT-AAA-GGA-G, Kif20a R: GAA-GCC-TTG-GAA-CAC-ACG-AGT-C, Kif20b F: GGA-TGA-CCT-AGA-CGT-GCT-TAC-C, Kif20b R: TCG-CTT-GAG-TGG-TAA-GGA-CAG-C, Nuf2 F: CCT-CTA-TGG-TCA-GAA-TGC-AGC-AG, Nuf2 R: ACT-GCT-TGA-ACT-CCT-CTC-GCT-C, Nusap1 F: TTC-CTC-CAA-GAG-GAA-GGC-TCT-C, Nusap1 R GGT-GTC-TTG-GTC-AGT-GAG-CAC-T, Top2a F: CAA-GCG-AGA-AGT-GAA-GGT-TGC-C, Top2A R: GCT-ACC-CAC-AAA-ATT-CTG-CGC-C, Cenpf F: GCA-CGA-CTT-CAG-TTA-CAG-GAG-C, Cenpf R: TGC-TTT-GGT-GTT-TTC-TCT-GTA-GTC, Ckap2 F: TAC-TGA-CCA-GCG-CAG-ATA-CAC-G, Ckap2 R: TCC-TTT-GCC-AGT-TCT-CCA-CTC-C, Ccna2 F GCC-TTC-ACC-ATT-CAT-GTG-GAT, Ccna2 R: TTG-CTC-CGG-GTA-AAG-AGA-CAG

| Name | Taqman Assay ID |
|---|---|
| Hsa-miR-127 | 000452 |
| sno135 | 001230 |
| Meg3 mouse | Mm00522599 |
| Meg3 human | Hs00292028 |
| Glul | Mm00725701 |
| APC total form | Mm00545877 |
| APC deleted form | Mm01130462 |
| human RIAN (MEG8) | Hs00419701_m1 |
| human MIRG (MEG9) | Hs01593046_s1 |
| mouse Mirg | Mm01335848 |
| human RTL-1 | Hs06637009_s1 |
| mouse RTL-1 | Mm02392620_s1 |
| mouse Rian | Mm01325842_g1 |
| 18S | 4308329 |

### ATAC-seq/ATAC-qPCR

For ATAC-seq experiments, 50,000 isolated hepatocytes were transposed for 30 min in 50 μL reaction mix containing 4.5 μL transposase (Illumina kit #FC-121-103) and 0.1% digitonin (adapted from ⁵). After transposition, the following steps were performed according to the initial protocol published by Buenrostro et al. ⁶. Omni-ATAC-seq was performed on frozen liver tumors according to Corces et al.⁷ (Supplementary Protocol 2). Firstly, nuclei were isolated from liver samples in 2 ml cold 1x Homogenization Buffer (2.1M Sucrose, Hepes 10 mM Buffer pH7,6, 2 mM EDTA pH8, 15 mM KCl, 10% Glycerol, 0.15 mM Spermine, 0.5 mM Spermidine) using an ultra-turrax for 4 min at speed 5. Nuclei were isolated after centrifugation at 20,000×g for 45 min and washing in 1 mL ATAC-RSB (10 mM Tris-HCl pH 7.4, 10 mM NaCl, 3 mM MgCl₂). Then, 50,000 nuclei were used for transposition for 30 min in 50 μL reaction mix containing 2.5 μL transposase (Illumina kit #FC-121-103), 0.01% digitonin and 0.1% Tween20. After transposition, the following steps were performed according to the initial protocol⁶. Libraries were controlled using a 2100 Bioanalyzer, and an aliquot of each library was sequenced at low depth onto a MiSeq platform to control duplicate level and estimate DNA concentration. Each library was then paired-end sequenced (2×100 bp) on a HiSeq instrument to get 40 million read pairs on average. As ATACseq libraries are composed in large part of short genomic DNA fragments, and in order to reduce costs, we next decided to sequence our recent libraries on a Nextseq instrument (2×38 bp). Our analysis showed that reducing read length to 38 bp does not affect mapping efficiency. Reads were first cleaned using trimmomatic (removing of adaptors and low-quality bases). Trimmed reads are then aligned to the mouse genome (mm9) using Bowtie2 with the parameter −X2000, and with 2-mismatches permitted in the seed (default value). The −X2000 option allows the fragments <2 kb to align. Duplicated reads were removed with picard-tools. Resulted bam datasets were then converted to BigWig, a coverage track adapted to visualize datasets in UCSC Genome Browser or IGV. Conversion was performed using bamCoverage command from deepTools with the parameters—binSize 10—normalizeUsing RPKM—extendReads. The parameter—normalizeUsing RPKM is used to normalize each dataset. We selected the normalization method based on RPKM (Reads Per Kilobase per Million mapped reads), which calculates the number of reads per bin/number of mapped reads (in millions). The parameter--extendReads allows the extension of reads to fragment size. The default value is estimated from the data (mean of the fragment size of all mate reads). Data are available in GSE206262.

In human HB, Single-nucleus Multiome ATAC+RNA-sequencing was performed by Integragen SA (Evry, France) on matched non-tumor livers (n=2) and hepatoblastomas (n=3) of two patients, following the Chromium Single Cell Multiome ATAC+Gene Expression protocol. We used 10× Genomics Cell Ranger ARC 2.0.0 to align snATAC-seq reads to the human genome (GrCh38/hg38).

For ATAC-qPCR experiments, 50,000 nuclei from isolated hepatocytes were used as previously for omni-ATAC protocol.

### Chromatin Immunoprecipitation (ChIP) and RNA Immunoprecipitation (RIP)

After perfusion, hepatocytes were washed in phosphate-buffered saline and centrifuged at 50×g for 3 min for subsequent experiments. Crosslinking was performed with 0.5% formaldehyde supplemented with 100 mM 4-(aminoethyl)-benzenesulfonyl fluoride (AEBSF) during 10 min and stopped with 125 mM glycine during 5 min. Ten million of cells were then lysed during 10 min at 4° C. in 50 mM Tris-HCl pH 7.5, 10 mM EDTA and 1% SDS supplemented with 100 mM AEBSF and protease inhibitors. Cells were then sonicated with a probe system in ChIP buffer (167 mM NaCl, 16.7 mM Tris-HCl pH 7.5, 1.2 mM EDTA, 1% Triton, 0.01% SDS) with a Bioruptor Plus (Diagenode) during 15 min at high frequency (30s on/30s off). Chromatin immunoprecipitation was performed overnight at 4° C. on 25 μg sonicated chromatin with 30 μl protein A/G dynabeads (v/v) with antibodies of interest: 10 μL β-catenin (BD biosciences 610154), 3 μL TCF-4 (Millipore 05-511), 10 μL H₃K₂₇Ac (Active Motif 39133), 5 μL H₃K₄me3 (Active Motif 39159), 10 μL H₃K₄me1 (Active Motif 39297), and 10 μL FoxM1 (Santa Cruz, sc-376471). After elution in 100 μl elution buffer (1%, SDS, 50 mM NaHCO₃) during 15 min at 65° C., the ChIP products were reverse-crosslinked with 10% Chelex at 100° C. for 10 min. For RIP experiments, FoxM1 immunoprecipitation was conducted as previously for ChIP. After overnight incubation, beads were washed with ChIP buffer five times and reverse crosslinked before RNA isolation by Trizol and MiRNeasy mini kit (Qiagen).

ChIPseq, RNAseq, smallRNAseq

ChIPseq, RNAseq and smallRNAseq experiments conducted on wild-type and Apc^{Δhep} mice have been previously published^{8,4} (Project: PRJNA150641 in ENA). RNAseq and smallRNAseq were performed on at least 4 samples from Apc^{Δhep} ROSA versus DLK1/DIO3^{ΔWRE} hepatocytes respectively with TruSeq Stranded after ribodepletion and TruSeq Small RNA on lug total RNA and sequenced with Nextseq 500 (150b). Fastq files were then aligned using STAR algorithm (version 2.7.6a), on the Ensembl Mus musculus GRCm38 reference, release 96. Reads were then count using RSEM (v1.3.1) and the statistical analyses on the read counts were performed with R (version 3.6.3) and the DESeq2 package (DESeq2_1.26.0) to determine the proportion of differentially expressed genes between two conditions.

We used the standard DESeq2 normalization method (DESeq2's median of ratios with the DESeq function), with a pre-filter of reads and genes (reads uniquely mapped on the genome, or up to 10 different loci with a count adjustment, and genes with at least 10 reads in at least 3 different samples). Following the package recommendations, we used the Wald test with the contrast function and the Benjamini-Hochberg FDR control procedure to identify the differentially expressed genes. R scripts and parameters are available on GitHub (https://github.com/BSGenomique/genomic-rnaseq-pipeline/releases/tag/v1.0420). For mir RNA-Seq data analysis Fastq files were uploaded on Qiagen geneglobe analysis software, for alignment and counting. Then, UMI matrix have been used as raw data for our R & DESeq2 pipeline (cf. RNA-Seq data analysis). Data are available in GSE206262.

### Chromosome Conformation Capture (3C)

Chromosome conformation capture assays were conducted as previously published^{9,10} Nuclei were extracted from 10 million isolated hepatocytes in 4 ml of Homogeniser Buffer (1.5M sucrose; 15 mM Hepes pH7.6; EDTA pH8 0.2 mM; KCl 60 mM, Spermine 0.15 mM and Spermidine 0.5 mM). Homogenisation is performed with an Ultra-turrax at speed 5 during 20 min on ice and solution centrifuged for 1 h at 20,000×g and 4° C. Pellets were then washed with 2 ml of wash buffer (10 mM Tris-HCl pH 7.4; 15 mM NaCl; 60 mM KCl; 0.15 mM spermine; 0.5 mM spermidine) and stored in glycerol buffer at −80° C. (40% glycerol; 50 mM Tris-HCl pH8.3; 5 mM MgCl₂; 0.1 mM EDTA). 3C experiments were conducted on 5 million nuclei with EcoRI digestion, product ligation and secondary XbaI digestion (N=5).

### Immunostaining/In Situ Hybridization (ISH)

Paraffin-embedded liver sections were treated and labeled with Ki67 and caspase 3 antibodies as previously described ⁴. For ISH, sections were deparaffinized and treated with 0.1 mg/ml proteinase K, then hybridized for 1 h at 55° C. with 100 nmol digoxigenin-labeled-LNA-scramble or LNA-127 (Exiqon) or a homemade probe against Meg3 in sense or antisense. Signals were detected with anti-digoxigenin (Roche) and Nitro BlueTetrazolium/5Bromo-4Chloro-3Indolyl phosphate (Roche).

### Western-Blot

Experiments were conducted as previously described on 20 μg total proteins ⁴. Incubations with primary antibodies were performed at 4° C. overnight. Anti-DLK1 was from Proteintech (reference 10636-1-AP, 1/1000), anti-Cyclin B1 from Cell Signaling (reference 4138, 1/200) and anti-cyclin A2 from Abcam (reference ab 32386, 1/200). Anti-β-actin was from Sigma-Aldrich (A5441, 1/5000)

### Statistical Analysis

We assessed the significance of differences between two groups of samples using Mann-Whitney tests. ANOVA was used to compare three groups of samples. p<0.05 was considered statistically significant. For human samples, difference in gene expression levels, in 2 or more than 2 groups, was tested using Wilcoxon or Kruskal-Wallis test, respectively. Correlation analysis was performed using Pearson r correlation when both variables were normally distributed with the assumptions of linearity and homoscedasticity or Spearman's rank-order correlation.

### Results

### The DLK1/DIO3 Locus is Over-Expressed in Hepatoblastoma

The expression of miRNAs as well as long non-coding RNAs, such as MEG3, RIAN, and MIRG, but also of the coding RNAs DLK1 and DIO3, was induced among all HB samples compared to their paired adjacent non-tumor tissues (n=24) (not shown). Interestingly, a strong correlation was seen between expression of the different RNAs produced from the locus-either lncRNAs or miRNAs (not shown)—supporting the idea of a global induction of this region in these tumor types. We were unable to correlate upregulation with CTNNB1 mutational status given the insufficient number of non-mutated tumors in this small cohort. In a larger cohort of HB (n=47)²² (not shown), induction of RIAN, RTL1, and MEG3 was confirmed in primary as well as recurrent HB compared to non-tumor livers (not shown). A strong correlation was also observed between RIAN and MEG3 but also between DIO3 and DIO3OS (not shown). DIO3 and DIO3OS were also highly expressed in the mesenchymal tumor subgroup, while RIAN was decreased and MEG3 was not found associated with any tumor subgroups (not shown). Importantly, we found a strong correlation between expression of RIAN or DIO3OS with CTNNB1-mutated HB (not shown). Therefore, deregulation of the DLK1/DIO3 locus appears frequent during HB development associated with CTNNB1 mutational status.

The Dlk1/Dio3 Locus is Induced after Sustained β-Catenin Activation in Mouse Livers and Tumors

We thus decided to investigate the relationship between the β-catenin and the Dlk1/Dio3 locus in mouse models recapitulating HB. Our team has created mouse models using both the Cre-Lox (Apc^{Δhep}) or CRISPR/Cas9 (β-cateninΔExon3) strategies that recapitulate liver cancer development with aberrant β-catenin activation. In both models, two tumor types can emerge with either good or poor differentiation features²¹; tumors with a well differentiated phenotype cluster with human HCC from the G5-G6 group, while less differentiated tumors cluster with human HB ²¹.

Shortly after β-catenin activation, Apc^{Δhep} hepatocytes overexpressed all coding and non-coding RNAs produced from the Dlk1/Dio3 locus except Dio3 compared to wild-type (wt) hepatocytes (raw data in ^{5,18}): Paternally-expressed DLK1 and RTL1 were overexpressed, as well as maternally-produced Meg3, Rian, Mirg and both miR-127/miR-136 and miR-379/miR-410 clusters (not shown). Induction of Meg3 and miR-127 was also confirmed in Apc^{Δhep} hepatocytes by in situ hybridization (ISH) (not shown), in which both RNAs colocalized. Meg3 and miR-127 upregulation was also observed by ISH in Apc^{Δhep} tumors in both well or poorly differentiated tumors (not shown). RT-qPCR analysis also showed induced Mirg, Rian, and miR-127 expression in Apc^{Δhep} tumors compared to their adjacent non-tumor tissues (not shown). This upregulation was stronger in well differentiated tumors for the three RNAs (not shown). It appears that, as for other metabolic β-catenin targets such as Glul or Cyp2e1, the expression of the Dlk1/Dio3 locus was higher in differentiated tumors, while Axin-2 and Ccnd1 were highly expressed in the more proliferative undifferentiated tumors, as we ²¹ and others²³ have previously shown (data not shown). It is noteworthy that strong correlations between Rian, Mirg, and miR-127 expression was found in Apc^{Δhep} tumors but also with Glul, a canonical β-catenin target gene in hepatocytes (not shown). Upregulation of Rian, Mirg, and miR-127 was also found in β-catenin^{ΔExon3} tumors by ISH (not shown) and by RT-qPCR, but to a lesser extent than in Apc^{Δhep} tumors and despite all tumors being well differentiated (mean fold upregulation of Rian: 7.2; Mirg: 3.7; miR-127:4.2 versus Rian: 47; Mirg: 74; miR-127:54 in Apc^{Δhep} tumors) (not shown). A strong correlation between Rian, Mirg, and miR-127 was also observed in β-catenin^{ΔExon3} tumors (not shown). This supports the hypothesis that the Dlk1/Dio3 locus is induced in a global manner in β-catenin driven liver tumors in mouse.

We injected a Cre-GFP adenovirus to sort GFP+hepatocytes and follow β-catenin driven reprogramming in hepatocytes by RNAseq during the earliest steps of liver tumorigenesis compared to non-activated GFP-hepatocytes (not shown). Figure S3A shows that several RNAs produced from the Dlk1/Dio3 locus were induced six days after injection, i.e., Rian, Mirg, and Rt11, but also pseudogenes such as B830012L14Rik and Gm37899. Interestingly, several other β-catenin positive targets also reached peak expression at day 6, including Glul, Lgr5, Cypla2, and Lect2 (not shown). There was a trend for all expressions to remain upregulated until day 15, with expression decreasing by day 21 but remaining higher than those observed in GFP-hepatocytes (except for Rt11 with expression drastically decreasing by day 15). These results indicate that the expression of the DLK1/DIO3 locus is not only correlated with the transcriptional program regulated by oncogenic β-catenin signaling, but is also highly deregulated at the earliest steps of liver tumorigenesis.

### TCF-4/β-Catenin Complexes Bind Upstream of Meg3 to Promote an Active Enhancer Site

Our next objective was to decipher how β-catenin activation promotes locus expression and if a direct regulation is involved. Firstly, our previously described ChIP-seq experiments⁵ in isolated Apc^{Δhep} hepatocytes showed that binding of TCF-4 was strongly increased in Apc^{Δhep} versus wild-type (wt) hepatocytes on a site named DLK1-WRE. This site is located upstream of the Ig-DMR and Meg3, and contains two WREs sites (not shown). Increased binding of TCF-4 and β-catenin was confirmed by ChIP-qPCR (not shown). In addition, an open chromatin configuration was observed around the DLK1-WRE site by both ATAC-seq experiments (not shown) and ATAC-qPCR experiments (not shown). All results were confirmed in β-catenin^{Δexon3} livers (not shown).

ChIP analysis of histone marks were subsequently conducted to precisely define the regulatory events occurring in the vicinity of the DLK1-WRE site. No significant differences were found in the H₃K₄me3 mark associated with actively transcribed promoter regions between the both models (not shown). These results indicate that this site does not act as a promoter region. However, H₃K₄me1 and H₃K₂₇Ac histone marks, typical of enhancers, were both found significantly increased at the DLK1-WRE site in Apc^{Δhep} hepatocytes compared to wt hepatocytes (not shown). Importantly, no modification in DNA methylation profiles, nor TCF-4 binding, were observed at the Ig-DMR regions in Apc^{Δhep} hepatocytes compared to wt hepatocytes by MeDIP-seq analysis (not shown ¹⁹). Interestingly, HNF4α, a key transcriptional factor involved in hepatocyte differentiation, is also able to bind at the Dlk1/Dio3 locus independently on TCF-4 binding at the DLK1-WRE site (not shown) and independent complexes (FIG. S4G). HNF4a binding is associated with locus transcription since Meg3 and miR-127 expression was impaired in HNF4α^{Δhep} hepatocytes (not shown) and consistently with previous data²⁴. This could participate to the higher expression observed in differentiated tumors, which kept expression of HNF4 a compared to undifferentiated tumors (not shown). In consequence, we have demonstrated here that an oncogenic activation of β-catenin signaling increases β-catenin/TCF-4 binding on the specific regulatory DLK1-WRE site, creating an enhancer site and favoring chromatin opening potentially prone to enhancing transcription of the entire DLK1/DIO3 locus.

Besides its transcriptional role, β-catenin is able to bridge two distal DNA regions by chromatin looping⁷ and the tridimensional structure of the DLK1/DIO3 locus is highly dynamic in correlation with its expression during embryonic development ^{25,26}. We thus performed a chromosome conformation capture (3C-qPCR) analysis centered on the DLK1-WRE site and covering the region between DLK1 and miR-136. The relative contact frequencies measured all along the locus allow to determine specific interactions: the higher the frequency, the closer the region of interest is relative to the DLK1-WRE site (not shown). As shown in wt hepatocytes (not shown), five major DNA loops with the DLK1-WRE site were occupied by low affinity TCF-4-binding and modified in response to β-catenin activation: one binding with a site upstream of DLK1, binding with two regions in the vicinity of the DLK1-WRE site (sites 1 and 2), binding within two sites downstream of the DLK1-WRE site, within the Ig-DMR (site 3) and within Meg3 (site 4). In Apc^{Δhep} hepatocytes (not shown), DLK1-WRE occupancy by TCF-4 resulted in DNA loop remodeling. Indeed, binding between the DLK1-WRE site and sites 1, 2, and 4 were lost, while looping within the Ig-DMR (site 3) was reinforced. A new loop between the DLK1-WRE site and the miR-136 region was also created (site 6). It is noteworthy that two of these loops coincide with two open chromatin regions observed by ATAC-seq in Apc^{Δhep} hepatocytes (sites 3 and 6 highlighted (pink) in FIG. 3A). Therefore, in Apc^{Δhep} hepatocytes, it appears that β-catenin/TCF-4 complexes bound at the DLK1-WRE site largely modify locus conformation to favor binding between coding regions located after the Ig-DMR instead of regions upstream of the Ig-DMR. This process could regulate the expression of non-coding RNAs encoded by the DLK1/DIO3 locus.

### β-Catenin/TCF-4 Binding at the DLK1-WRE Site is Required for an Optimal Enhancer Activity, Chromatin Remodeling, and Subsequent Maximal Transcription

To decipher if the DLK1-WRE site is the key region underlying its regulation via β-catenin and to precise the roles of the DLK1/DIO3 locus during liver tumorigenesis, we used CRISPR-Cas9 machinery to specifically remove the DLK1-WRE site. This deletion was called DLK1/DIO3^{ΔWRE} (not shown). We designed two small guide RNAs (sgRNAs) to frame the TCF-4 binding site without potential off-targets. Then, sgRNAs were integrated into plasmids containing the saCas9 sequence and inverted terminal repeats, allowing in vivo editing using AAV8 particles²⁷. We validated these constructs first in vitro by stable transfection and clonal selection in the murine hepatoma Hepa1-6 cell line with a short activating deletion in exon 3 of Ctnnb1. We could notice that DNA editing (not shown) was successfully associated with a decrease in Rian, Mirg, Meg3, and Rt11 expression by RT-qPCR (not shown), as well as deregulated DLK1 protein expression by Western blotting (not shown). We then produced AAV2/AAV8 particles containing each sgRNA under the control of the hepato-specific promoter TBG²⁷ and tested them in vivo one month after the retroorbitary injection into wt and Apc^{Δhep} mice (not shown); a one-month time frame has been previously identified as optimal for hepatocyte-specific CRISPR/cas9 editing²¹. We were indeed able to target approximately 30% of total liver cells-which is equivalent to 50% of hepatocytes (not shown)-without editing in non-parenchymal cells (not shown). Importantly, CRISPR-Cas9 editing of the DLK1-WRE site resulted in a drastic decrease in Rian and Mirg expression in Apc^{Δhep} DLK1/DIO3^{ΔWRE} hepatocytes (not shown).

TCF-4 binding was significantly reduced in Apc^{Δhep}DLK1/DIO3^{ΔWRE} hepatocytes as expected (not shown). More interestingly, both H₃K₄me1 and H₃K₂₇Ac marks associated with active enhancers were impaired in Apc^{Δhep}DLK1/DIO3 hepatocytes compared to Apc^{Δhep} hepatocytes (not shown). Chromatin opening at the DLK1-WRE site was also significantly reduced in Apc^{Δhep}DLK1/DIO3^{ΔWRE} hepatocytes (not shown). Finally, 3C experiments conducted in Apc^{Δhep}DLK1/DIO3^{ΔWRE} hepatocytes showed that the chromatin interactions with the DLK1-WRE site-containing region had comparable conformations to those seen in wt hepatocytes (not shown): sites 1, 2 and 4 get closer to the region containing the DLK1-WRE site while site 3 (in the Ig-DMR) and 6 (near miR-136) was further away. A new weak interaction between the DLK1-WRE site and site 5, located downstream of Meg3, was revealed in Apc^{Δhep}DLK1/DIO3^{ΔWRE} hepatocytes.

In all, these data indicate that the chromatin and epigenetic modifications observed in the Dlk1/Dio3 locus in Apc^{Δhep} hepatocytes with the subsequent induction of the Dlk1/Dio3 locus itself, were specifically driven by TCF-4/β-catenin binding at the DLK1-WRE site. Our CRISPR/cas9 system efficiently impaired transcription of the Dlk1/Dio3 locus in vivo and thus allow next to study of the role of this locus during β-catenin-dependent tumorigenesis.

### Editing of the DLK1-WRE Site Impairs the Pro-Tumorigenic Capacities of β-Catenin-Mutated Hepatic Cancer Cell Lines

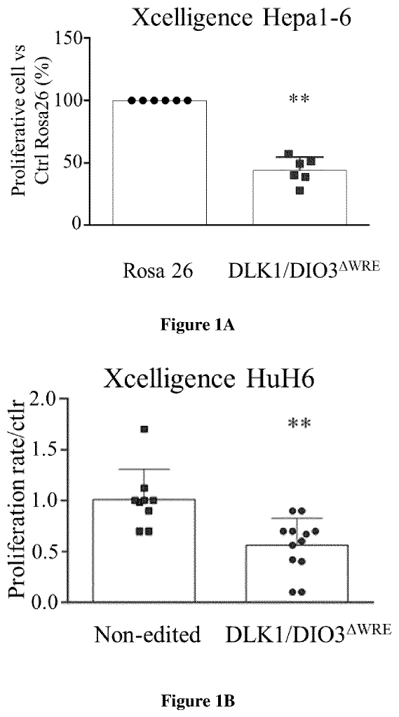

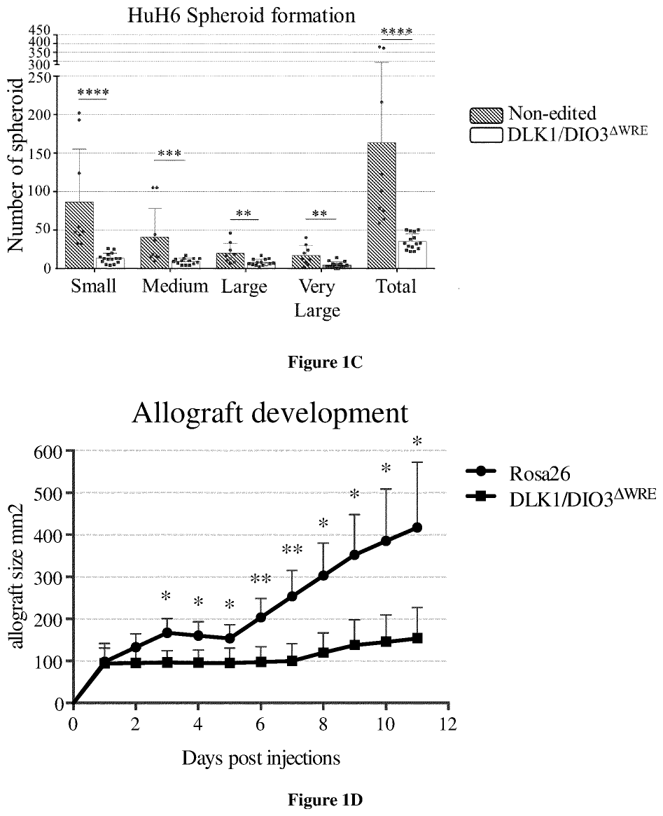

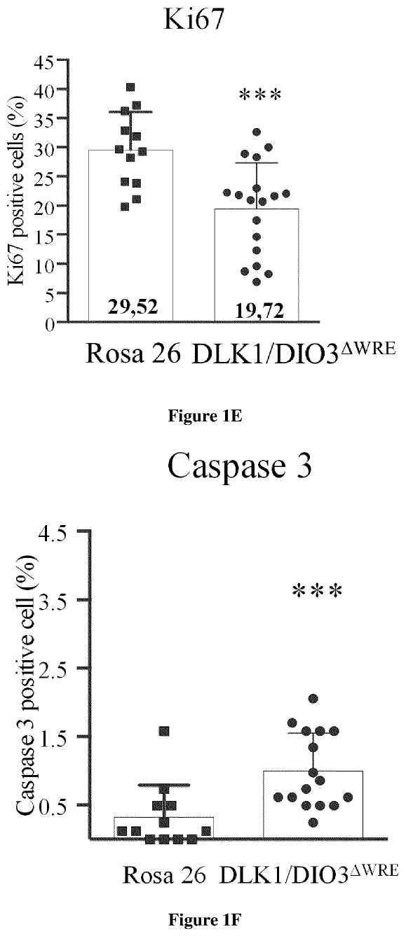

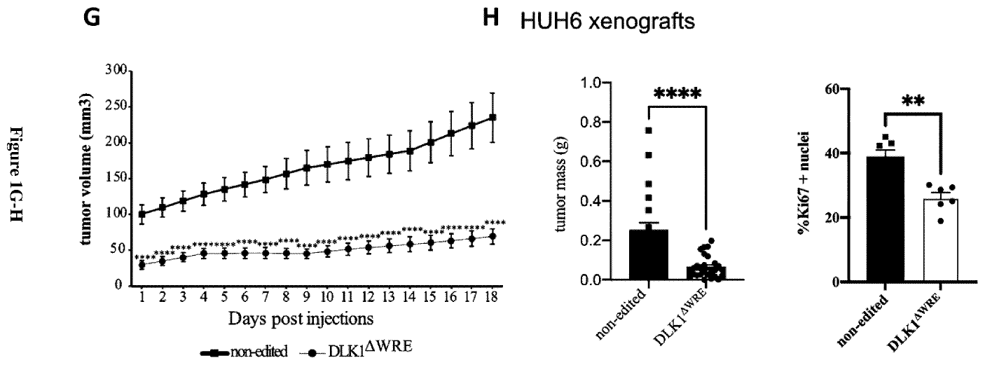

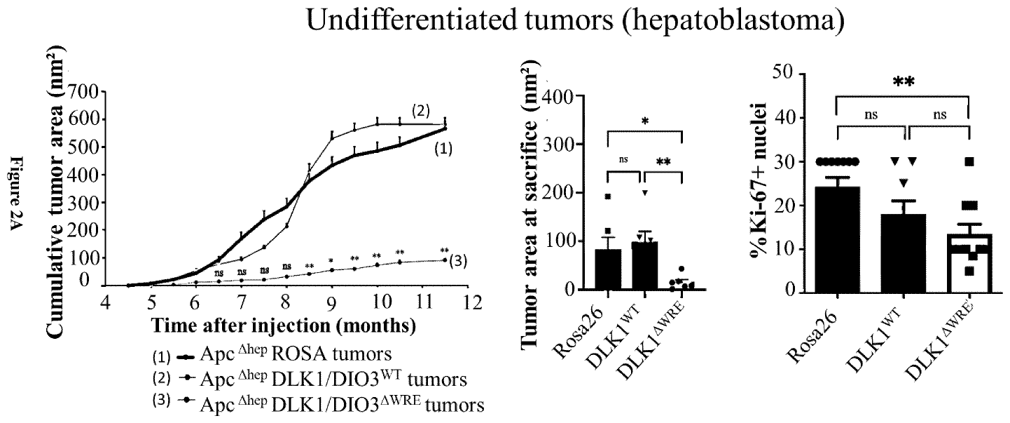

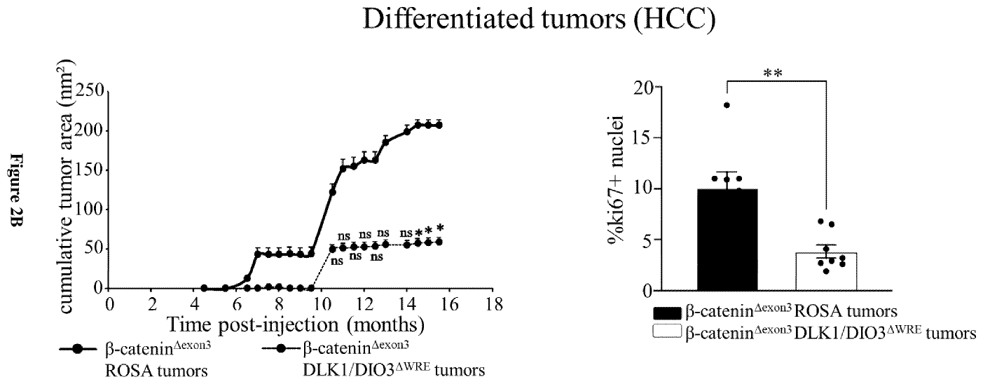

We firstly assessed the effect of DLK1-WRE site editing in vitro in stable clones generated using either Rosa26 or DLK1/DIO3^{ΔWRE} murine hepatoma Hepa1-6 cells. DLK1/DIO3^{ΔWRE} cells were less proliferative than the Rosa26 cells according to XCelligence monitoring (FIG. 1A). This was accompanied with a significant increase in the number of DLK1/DIO3^{ΔWRE} cells in G2/M phase (not shown) together with a decrease in cyclin B1 protein level, a protein required for mitotic initiation (not shown). Consistent with in vitro data, DLK1/DIO3^{ΔWRE} cells exhibited decreased tumorigenic capacity compared to Rosa26 cells after subcutaneous allografting into Nu/Nu mice; tumor progression was significantly slower for DLK1/DIO3^{ΔWRE} cells (FIG. 1D). In line, DLK1/DIO3^{ΔWRE} tumors had a mean volume of approximately 150 mm3 versus 400 mm3 for Rosa26 tumors, with a 4-fold lower weight for DLK1/DIO3^{ΔWRE} tumors at the time of sacrifice. Impaired DLK1/DIO3^{ΔWRE} tumor progression in Nu/Nu mice was consistent with less Ki-67 positive cells (FIG. 1E) but more cells harboring cleaved caspase-3 (FIG. 1F) compared to Rosa26 tumors. The level of FADD, a potential target of miR-134 in the algorithm of prediction Dianalab, was consistently found increased in DLK1/DIO3^{ΔWRE} tumors at the mRNA and protein level (data not shown). In publicly accessible ChIP-sequencing data, a TCF-4 binding site has also been reported upstream of MEG3 (chr14: 101,283,029-101,284,356 in UC Davis GSM782122) in human hepatoblastoma-like HepG2 cells (not shown). These cells display an activating deletion in exon 3 of CTNNB1. Importantly, snATAC-seq experiments realized on HB samples harboring CTNNB1 mutations showed that chromatin was opened at this site in tumors compared to non-tumor tissues (not shown). This suggests an evolutionary conserved mechanism for β-catenin-driven regulation of the DLK1/DIO3 locus. Therefore, we generated DLK1/DIO3^{ΔWRE} hepatic cancer cells of human origin. We chose hepatoblastoma-like Huh6 cells with an activating mutation in the CTNNB1 gene. As previously described, we designed different sgRNAs to delete the DLK1-WRE site and obtained three different stable clones with efficient editing, of either or both WREs (not shown). Editing efficiently impaired the expression of RIAN, MIRG, MEG3, and RTL-1 in Huh6 cells (not shown) and proliferation of all DLK1/DIO3^{ΔWRE} Huh6 clones was lower than for control cells (FIG. 1B). We also observed a significant increase in the number of cells in G2/M phase for Huh6 DLK1/DIO3^{ΔWRE} cells compared to control cells (not shown), supporting the idea that preventing the binding of TCF-4/β-catenin on the DLK1-WRE site impairs mitotic entry. Secondly, considering that Huh6 cells, contrary to Hepa1-6 cells, have the capacity to efficiently form spheroids, we assessed if the DLK1-WRE site editing changed spheroid formation capacity. FIG. 1C showed that DLK1/DIO3^{ΔWRE} cells formed a significantly reduced number of spheroids in total compared to control cells. DLK1/DIO3^{ΔWRE} HuH-6 cells exhibited decreased tumorigenic capacity compared to control cells after subcutaneous allografting into Nu/Nu mice; tumor progression was significantly slower for DLK1/DIO3^{ΔWRE} cells (FIG. 1G). In line, DLK1/DIO3^{ΔWRE} tumors had 4-fold lower volume, lower weight at the time of sacrifice together with low number of Ki-67+tumor cells (FIG. 1G-H).

All these data strongly defend the hypothesis that the DLK1/DIO3 locus contributes to the pro-tumorigenic capacities of transformed hepatic cancer cells harboring β-catenin mutations by enhancing their proliferation and stemness both in vitro and in vivo.

### DLK1-WRE Site Editing in Apc^{Δhep} Hepatocytes Primarily Affects Regulators of Mitotic Entry and Progression

After these extensive studies in cancer cell lines, we assessed the consequences of DLK1-WRE site editing in vivo in the Apc^{Δhep} mice (not shown). Apc^{Δhep} DLK1/DIO3^{ΔWRE} mice demonstrated significantly reduced hepatomegaly versus Apc^{Δhep} mice²⁸, with a mean liver-to-body weight ratio at 7.6%, versus 8.3% in Apc^{Δhep} mice. Wt mice exhibited an approximate 5% liver-to-body ratio as expected (not shown). Reduced liver size was associated with a significant decrease in the numbers of Ki-67 positive hepatocytes in Apc^{Δhep} DLK1/DIO3^{ΔWRE} livers (not shown).

To identify the downstream molecular mechanisms resulting from DLK1-WRE site editing, we performed both RNAseq and small-RNAseq on Apc^{Δhep} Rosa26 and Apc^{Δhep} DLK1/DIO3^{ΔWRE} hepatocytes. Firstly, editing of the DLK1-WRE site resulted in a significant decrease in the majority of miRNAs and lncRNAs produced from the locus in Apc^{Δhep} DLK1/DIO3^{ΔWRE} compared to Apc^{Δhep} Rosa26 hepatocytes (not shown). This ultimately confirmed that the entire locus is transcriptionally regulated by TCF-4/β-catenin binding in the event of the oncogenic activation of this pathway in Apc^{Δhep} hepatocytes. More importantly, several genes implicated in cell proliferation and division were among the top 50 genes deregulated in Apc^{Δhep} DLK1/DIO3^{ΔWRE} hepatocytes versus Apc^{Δhep} Rosa26. For example, the following genes were downregulated: Ccna2, Ccnb1, Kif20a, Kif20b, Ckap2, Top2a, Cdc2 Racgap1, Kif4a, Hmmr, Nusap1, and Nuf2 (not shown). Interestingly, all these actors were found upregulated in Apc^{Δhep} hepatocytes compared to wt hepatocytes (not shown). In a more global analysis, the STRING database of significantly deregulated RNAs established hub genes associated with mitotic sister chromatid segregation and cyclin A/B1/B2 associated events during G2/M transition (not shown). Gene set enrichment analysis of the downregulated RNAs in Apc^{Δhep} DLK1/DIO3^{ΔWRE} hepatocytes identified GO terms related to the microtubule cytoskeleton, mitotic spindle, cytokinesin complex, and the cyclin B1/CDK1 complex (not shown). RT-qPCR analyses shown in FIG. 5E on a larger sample collection validated the deregulations of major cell cycle regulators including Nuf2, Top2a, Kif20b and Nusap1 in Apc^{Δhep} DLK1/DIO3^{ΔWRE} hepatocytes versus Apc^{Δhep} Rosa26 hepatocytes.

FoxM1, a typical proliferation-associated transcription factor, regulates the expression of genes involved in G2/M-transition and M-phase progression²⁹. The expression of such genes (Kif20a, Ccna2, Cdc2, or Cenpf) was deregulated in DLK1/DIO3^{ΔWRE} hepatocytes (not shown). Therefore, we questioned if alterations in FoxM1 expression or activity could be involved in the gene expression deregulations observed in Apc^{Δhep} DLK1/DIO3^{ΔWRE} hepatocytes. We did not observe any difference in Foxm1 expression at the mRNA level (not shown) or nuclear localization (not shown) between Apc^{Δhep} DLK1/DIO3^{ΔWRE} hepatocytes and Apc^{Δhep} Rosa26 hepatocytes. We then explored by ChIP-PCR if FoxM1 binding was affected on the promoter regions of some targets in Apc^{Δhep} DLK1/DIO3^{ΔWRE} hepatocytes compared to Apc^{Δhep} Rosa26 hepatocytes. FIG. 6D showed that FoxM1 binding was decreased on Ccna2, Kif20a and cdc2 promoters in Apc^{Δhep} DLK1/DIO3^{ΔWRE} hepatocytes compared to Apc^{Δhep} Rosa26 hepatocytes. By FoxM1 immunoprecipitation, we could also observe that Meg3 was associated with FoxM1 in Apc^{Δhep} Rosa26 hepatocytes, while this association is undetectable in Apc^{Δhep} DLK1/DIO3^{ΔWRE} hepatocytes (not shown). In all, these data support the theory that the β-catenin/Meg3/FoxM1 axis plays a pivotal role in preneoplastic Apc^{Δhep} hepatocyte proliferation.

DLK1-WRE Site Editing Impairs Apc^{Δhep} and β-Catenin^{Δexon3} Tumor Progression

To finish, we studied the effect of DLK1-WRE site editing on the development of Apc^{Δhep} and β-catenin^{Δexon3} tumors. Follow-up of tumor development in our transgenic mouse models was performed by ultrasonography every two weeks as previously described^{18,19} (not shown). Firstly, follow-up demonstrated no significant difference in tumor progression in Apc^{Δhep} DLK1/DIO3^{ΔWRE} mice compared to Apc^{Δhep} Rosa26 mice (not shown). Nonetheless, it appeared that approximately 44% of tumors (8/18) were not DLK1-WRE site-edited at the time of sacrifice (Apc^{Δhep} DLK1/DIO3WT), while the ten others present different percentages of edition (not shown). Retrospective analysis of ultrasonography measurements demonstrated that Apc^{Δhep} DLK1/DIO3^{ΔWRE} tumor progression was reduced as early as their detection compared to Apc^{Δhep} Rosa26 tumors (not shown), in accordance with lower cumulative tumor areas at sacrifice (not shown). It was noteworthy that Apc^{Δhep} DLK1/DIO3WT tumor size and progression were significantly impaired compared to Apc^{Δhep} Rosa26 tumors, suggesting that the surrounding Apc^{Δhep} DLK1/DIO3^{ΔWRE} non-tumor tissues could influence tumor progression (not shown). The immunohistochemical analysis of these tumors showed that this experiment resulted in a vast majority of differentiated tumors (27/32) (not shown). As previously mentioned, Rian and Mirg expressions were the highest in differentiated Apc^{Δhep} tumors (not shown). We did not observe a significant reduction in Rian and Mirg expression in differentiated Apc^{Δhep} DLK1/DIO3^{ΔWRE} tumors compared to both Apc^{Δhep} DLK1/DIO3WT and Apc^{Δhep} Rosa26 tumors (not shown). Accordingly, we did not identify any significant differences in the numbers of Ki67+tumor cells (not shown) and in the levels of Mki67, Ccna2, Top2a and Kif20a (not shown). This is likely due to the insufficient editing and overexpression of the Dlk1/Dio3 locus in Apc^{Δhep} DLK1/DIO3^{ΔWRE} tumors (not shown).

In HB-like tumors (FIG. 7A), approximately 61% of tumors were edited (11/18) (not shown) and markedly expressed lower expression of Rian and Mirg compared to Apc^{Δhep} DLK1/DIO3 WT tumors (not shown). Follow-up demonstrated no significant decrease in tumor progression in Apc^{Δhep} DLK1/DIO3^{ΔWRE} and Rosa26 mice (not shown). Retrospective analysis of ultrasonography measurements demonstrated that Apc^{Δhep} DLK1/DIO3^{ΔWRE} tumors exhibited a lower progression rate and thus lower cumulative tumor area than Apc^{Δhep} DLK1/DIO3 WT tumors (FIG. 2A). This was consistent with a decreased number of Ki-67 positive tumor cells in Apc^{Δhep} DLK1/DIO3^{ΔWRE} tumors compared to Apc^{Δhep} DLK1/DIO3 WT tumors developing in the same non-tumor environment (FIG. 2A), but also in comparison with Apc^{Δhep} Rosa26 tumors (FIG. 2A). A decrease in mki67, ccna2, Nuf2, Top2a, Kif20b, Ckap2, Cenpf, and Nusap1 was observed in Apc^{Δhep} DLK1/DIO3^{ΔWRE} tumors compared to Apc^{Δhep} Rosa26 tumors (not shown).

The same experiments conducted in the β-catenin^{Δexon3} model²¹ (not shown) resulted exclusively in the development of well-differentiated β-catenin^{Δexon3} tumors (13/13) (not shown). We show that sustained β-catenin activation in β-catenin^{Δexon3} tumors leads to low expression of Rian, Mirg, and miR-127. It appeared that editing of the DLK1-WRE site was sufficient for impairing expression of Rian, Mirg, and miR-127 in β-catenin^{Δexon3} DLK1/DIO3^{ΔWRE} tumors (not shown), and only two tumors were non edited (β-catenin^{Δexon3} DLK1/DIO3WT). During ultrasound monitoring, β-catenin^{Δexon3} DLK1/DIO3^{ΔWRE} tumors exhibited lower progression rates compared to β-catenin^{Δexon3} Rosa26 tumors (FIG. 2B). This was consistent with a decrease in Ki-67 positive staining (FIG. 2B) and mRNA levels in β-catenin^{Δexon3} DLK1/DIO3^{ΔWRE} tumors (not shown). Cell cycle markers such as Top2a, Kif20b and Ckap2 were decreased in β-catenin^{Δexon3} DLK1/DIO3^{ΔWRE} tumors compared to β-catenin^{Δexon3} Rosa26 tumors (not shown).

In all, these results imply that the extinction efficiency depends on the activation level of the DLK1/DIO3 locus in tumors but also in the surrounding tissues. We thus decided to explore expression of the DLK1/DIO3 locus in human HCC and in non-tumor tissues. HCCs overexpressing MEG3, RIAN, and DIO3 primarily belonged to the G1 tumor subgroup characterized by frequent BAP1 mutations and overexpression of other imprinted genes³⁰ (not shown). In addition, CTNNB1-mutated HCCs showed a lower expression of the DLK1/DIO3 locus compared to non-mutated HCCs (not shown). Overall, the expression of MEG3, RIAN, DIO3, and DIO3OS was significantly down-regulated in HCCs compared to non-tumor liver tissues with an important disparity between tumors (not shown) (N=19831). In fact, MEG3, RIAN, and RTL1 were found overexpressed in non-tumor tissues as compared to healthy livers (not shown), suggesting that liver disease and cirrhosis can also induce the DLK1/DIO3 locus. Similarly in mice, Apc^{Δhep} mice fed a choline-deficient diet known to promote fatty livers expressed higher levels of Rian and Meg3 in non-tumor livers compared to Apc^{Δhep} mice fed a chow-diet group (FIG. S8E).

Altogether, our results show that the DLK1/DIO3 locus strongly affects β-catenin-dependent cell proliferation and division events, particularly those involved in G2/M progression, in vitro in transformed cells, but also in vivo in preneoplastic conditions and during tumor progression, both in HB-like and HCC tumors with moderate activation of the DLK1/DIO3 locus.

### Discussion

Imprinted loci orchestrate cellular plasticity in both embryonic development and adult tissues, via their dynamic monitoring, thus constituting major players in cell reprogramming during cancer initiation and progression. Among such imprinted loci, the correct dosage of the DLK1/DIO3 locus, also known as the 14q32.2 cluster, appears crucial for cell proliferation, senescence, and metabolic adaptation in cells of different origins¹² and notably from the liver³². In HCCs, expression of the DLK1/DIO3 locus is either increased or impaired depending on the associated etiology^{33,34} and the DLK1/DIO3 locus also constitutes a preferential site of viral insertion promoting HCCs^{35,36}. Deregulation of this genomic region has also been found associated with poor prognosis in HB¹⁶. In our present study, including two cohorts of patients with HB, we found that coding and non-coding RNAs produced from the DLK1/DIO3 locus are highly expressed in tumors. Additionally, we found a strong correlation between expressions of all RNAs and miRNAs produced from this locus. In accordance, we observed a correlation between CTNNB1 mutations and the upregulation of RIAN or DIO3OS, as supported by the recent work of Carrillo-Reixach and colleagues¹⁷. In adult HCCs, RNAs from the DLK1/DIO3 locus were induced in the G1 tumor subgroup with overexpression of genes controlled by parental imprinting and thus correlated more with BAP I than CTNNB1 mutations. Our present work also supports the hypothesis that deregulation of the DLK1/DIO3 locus is a frequent event underlying HCC pathogenesis. Induction in the DLK1/DIO3 locus additionally occurring in diseased livers since non-tumor tissues also harbored higher levels of MEG3, RIAN, and RTL1 compared to healthy livers. Our results are consistent with the increased MEG3 expression found in fibrosis and non-alcoholic steatohepatitis cirrhosis specimens³⁷.

Considering that HCC emerges on cirrhotic livers in 80% of cases, DLK1/DIO3 locus induction in non-tumor tissues could be associated with concomitant reprogramming of the tumor microenvironment. Accordingly, we observed a lower tumor progression rate for Apc^{Δhep} DLK1/DIO3^{WT} HCCs developing in Apc^{Δhep} DLK1/DIO3^{ΔWRE} livers versus Apc^{Δhep} Rosa26 HCCs, arguing that impairment of the DLK1/DIO3 locus in non-tumor tissues could modulate tumor progression. The expression of the DLK1/DIO3 locus is induced in response to several stresses, particularly in hepatocytes under metabolic disorders³⁸, but also in immune cells in response to lipopolysaccharide ³⁹ or during lupus ⁴⁰. Investigating this hypothesis, we found a significant induction of the locus in different mouse models of stress and inflammation (partial hepatectomy, methionine choline-deficient diet, or binge drinking). In addition, we observed that non-tumor tissues from Apc^{Δhep} mice fed a choline-deficient diet, known to promote fatty livers, expressed higher levels of Rian and Meg3 compared to non-tumor tissues in the chow-diet group. Together, these observations could support the idea of the great potential for impairing the DLK1/DIO3 locus in diseased livers. RNAs produced from the DLK1/DIO3 locus have been reported as keys regulators of macrophage activation and polarization^{41,42} opening new perspectives for the oncogenic role of this locus during liver tumorigenesis, that remain to be studied in our models.

Here, we identified the regulatory region activated by β-catenin/TCF-4 complexes and determined how this region promotes transcription of the DLK1/DIO3 locus in cancer. We then confirmed the functionality of the DLK1/DIO3 locus by AAV8-CRISPR/cas9 constructs in vitro as well as in vivo. This transcriptional regulation concerns the entire locus, coding but also non-coding RNAs (both mi- and lncRNAs), and depends on the direct binding of β-catenin/TCF-4 complexes at the DLK1-WRE site. The binding site becomes an active enhancer in response to oncogenic activation of β-catenin signaling, but this site is also engaged in chromatin remodeling and long-range chromatin interactions. Importantly, the binding of β-catenin/TCF-4 complexes is a prerequisite for epigenetic and chromatin landscape remodeling. These events bridge the enhancer region with other regulatory regions in the locus, notably regulatory sites at the Ig-DMR region, promoting in turn transcription of the entire region. This DNA looping role for β-catenin/TCF-4 complexes echoes two works published by Yochum et al. in colon cancer. The authors show how β-catenin/TCF-4 complexes coordinate chromatin looping at an enhancer site upstream of MYC, a canonical β-catenin target in the colon but not in the liver^{43,44}. In consequence, it appears that, depending on the tissue and the cell-type, β-catenin/TCF-4 complexes could transmit different oncogenic signals depending on the tissues and the cells at play through chromatin remodeling at distinct oncogenic actors.

Furthermore, this regulatory mechanism is reminiscent of events occurring at other imprinted loci during embryonic development, for which chromatin looping and enhancer-promoter bridging is a way to escape from silencing^{45,46} This includes the DLK1/DIO3 locus for which the Ig-DMR region harbors enhancer activity on its maternal allele⁴⁷. Unfortunately, our transgenic mouse models render impossible the study of if β-catenin/TCF-4-driven enhancer activity is specific to the maternal or paternal allele or both. However, no changes in DNA methylation status at the Ig-DMR regions were observed in Apc^{Δhep} hepatocytes when compared to wt hepatocytes using MeDIP-seq.

Besides increasing knowledge on the regulation of the DLK1/DIO3 locus, our editing strategy has also deciphered the pro-tumorigenic events subsequent to β-catenin-driven activation of the DLK1/DIO3 locus. In both Apc^{Δhep} and β-catenin^{ΔExon3} models, DLK1/DIO3^{ΔWRE} tumors exhibited a slower progression rate, particularly those with undifferentiated features similar to HB²¹. Regarding our experiments on two different liver cancer mouse models, it appears that inhibition of RNAs from the DLK1/DIO3 locus following DLK1-WRE site editing and inhibition of tumor progression are highly dependent on tumor DLK1/DIO3 locus level. In both HB-related tumors and differentiated β-catenin tumors, in which the expression of RNAs from the locus is lower, progression of DLK1/DIO3^{ΔWRE} tumors was significantly impaired. Our different data from DLK1/DIO3^{ΔWRE} hepatocytes and transformed cells revealed that inhibition of cell proliferation and tumor progression were associated with a decrease in the mRNA levels of several actors involved in cytokinesis and G2/M phase transition in both preneoplastic hepatocytes and tumors. Interestingly, a hub between actors in cell cycle or cell division with miRNAs produced from the DLK1/DIO3 locus has also been inferred by others following comprehensive bioinformatics approaches conducted on high-throughput datasets on HB⁴⁸. Here, our study shows that the effects of the DLK1/DIO3 locus-dependent cell proliferation and G2/M progression in preneoplastic hepatocytes is driven by β-catenin through its direct regulatory role on the locus enhancer region and subsequent redistribution of FoxM1 on the promoter regions of actors involved in cell cycle progression. Meg3 appears as a good candidate for FoxM1 guidance on the promoter regions of FoxM1-regulated genes to regulate their activation, since we did not identify any binding between FoxM1 and Rian or Mirg (FIG. S9). Using two transformed cell lines with activating mutations of Ctnnb1/CTNNB1, hepatoma Hepa1-6 cells and hepatoblastoma-like Huh6 cells, we confirmed a lower proliferation rate for DLK1/DIO3^{ΔWRE} cells together with arrest in G2/M phase; the cell phase during which the levels of β-catenin and its regulated gene repertoire rise to a peak⁴⁹.

Altogether, our results unveil how a sustained activation of β-catenin signaling can remodel the epigenetic and chromatin landscape of one of its key oncogenic targets with a subsequent effect on proliferative gene signatures. We put forward the DLK1-WRE site as a potent strategy for specific inhibition of the entire DLK1/DIO3 locus in liver tumors harboring activating β-catenin signaling mutations.

### CONCLUSION

In conclusion, our present work unveils that an aberrant activation of β-catenin in preneoplastic cells remodels a regulatory region upstream of the DLK1/DIO3 locus with active enhancer properties and capable of DNA contacts at distance leading to its entire transcription. This subsequently modifies FoxM1 distribution on the promoter region of actors involved in cell cycle progression leading to cell proliferation. These results support the key role of the DLK1/DIO3 locus during early steps of β-catenin-driven tumorigenesis through events occurring in hepatocytes but also suggest an involvement of the immune microenvironment to sustain hepatocyte transformation. This point appears of particular interest to understand why tumors with CTNNB1 mutations present a peculiar immune contexture. More importantly, our results demonstrate that disrupting the β-catenin/TCF-4 binding site located upstream of Meg3 in the DLK1/DIO3 locus represents a very interesting approach for the treatment of liver cancers.

### REFERENCES

Throughout this application, various references describe the state of the art to which this invention pertains. The disclosures of these references are hereby incorporated by reference into the present disclosure.
1. de La Coste, A, Romagnolo, B, Billuart, P, Renard, C A, Buendia, M A, Soubrane, O et al. Somatic mutations of the beta-catenin gene are frequent in mouse and human hepatocellular carcinomas. Proc Natl Acad Sci USA 1998; 95:8847-8851
2. Wei, Y, Fabre, M, Branchereau, S, Gauthier, F, Perilongo, G & Buendia, M A. Activation of beta-catenin in epithelial and mesenchymal hepatoblastomas. Oncogene 2000; 19:498-504
3. Hirsch, T Z, Pilet, J, Morcrette, G, Roehrig, A, Monteiro, B J E, Molina, L et al. Integrated Genomic Analysis Identifies Driver Genes and Cisplatin-Resistant Progenitor Phenotype in Pediatric Liver Cancer. Cancer Discov 2021; 11:2524-2543
4. Cadigan, K M & Waterman, M L. TCF/LEFs and Wnt signaling in the nucleus. Cold Spring Harb Perspect Biol 2012; 4
5. Gougelet, A, Torre, C, Veber, P, Sartor, C, Bachelot, L, Denechaud, P D et al. T-cell factor 4 and beta-catenin chromatin occupancies pattern zonal liver metabolism in mice. Hepatology 2014; 59:2344-2357
6. Gougelet, A & Colnot, S. A Complex Interplay between Wnt/beta-Catenin Signalling and the Cell Cycle in the Adult Liver. International journal of hepatology 2012; 2012:816125
7. Mosimann, C, Hausmann, G & Basler, K. Beta-catenin hits chromatin: regulation of Wnt target gene activation. Nature reviews. Molecular cell biology 2009; 10:276-286
8. Schulze, K, Imbeaud, S, Letouze, E, Alexandrov, L B, Calderaro, J, Rebouissou, S et al. Exome sequencing of hepatocellular carcinomas identifies new mutational signatures and potential therapeutic targets. Nat Genet 2015; 47:505-511
9. Riou, R, Ladli, M, Gerbal-Chaloin, S, Bossard, P, Gougelet, A, Godard, C et al. ARIDIA loss in adult hepatocytes activates beta-catenin-mediated erythropoietin transcription. Elife 2020; 9
10. Nagae, G, Yamamoto, S, Fujita, M, Fujita, T, Nonaka, A, Umeda, T et al. Genetic and epigenetic basis of hepatoblastoma diversity. Nat Commun 2021; 12:5423
11. Cairo, S, Armengol, C, De Reynies, A, Wei, Y, Thomas, E, Renard, C A et al. Hepatic stem-like phenotype and interplay of Wnt/beta-catenin and Myc signaling in aggressive childhood liver cancer. Cancer cell 2008; 14:471-484
12. da Rocha, S T, Edwards, C A, Ito, M, Ogata, T & Ferguson-Smith, A C. Genomic imprinting at the mammalian Dlk1-Dio3 domain. Trends in genetics: TIG 2008; 24:306-316
13. Benetatos, L, Hatzimichael, E, Londin, E, Vartholomatos, G, Loher, P, Rigoutsos, I et al. The microRNAs within the DLK1-DIO3 genomic region: involvement in disease pathogenesis. Cellular and molecular life sciences: CMLS 2013; 70:795-814
14. Huang, J, Zhang, X, Zhang, M, Zhu, J D, Zhang, Y L, Lin, Y et al. Up-regulation of DLK1 as an imprinted gene could contribute to human hepatocellular carcinoma. Carcinogenesis 2007; 28:1094-1103
15. Lopez-Terrada, D, Gunaratne, P H, Adesina, A M, Pulliam, J, Hoang, D M, Nguyen, Y et al. Histologic subtypes of hepatoblastoma are characterized by differential canonical Wnt and Notch pathway activation in DLK+ precursors. Hum Pathol 2009; 40:783-794
16. Honda, S, Chatterjee, A, Leichter, A L, Miyagi, H, Minato, M, Fujiyoshi, S et al. A MicroRNA Cluster in the DLK1-DIO3 Imprinted Region on Chromosome 14q32.2 Is Dysregulated in Metastatic Hepatoblastomas. Front Oncol 2020; 10:513601
17. Carrillo-Reixach, J, Torrens, L, Simon-Coma, M, Royo, L, Domingo-Sabat, M, Abril-Fornaguera, J et al. Epigenetic footprint enables molecular risk stratification of hepatoblastoma with clinical implications. J Hepatol 2020; 73:328-341
18. Gougelet, A, Sartor, C, Bachelot, L, Godard, C, Marchiol, C, Renault, G et al. Antitumour activity of an inhibitor of miR-34a in liver cancer with beta-catenin-mutations. Gut 2016; 65:1024-1034
19. Gougelet, A, Sartor, C, Senni, N, Calderaro, J, Fartoux, L, Lequoy, M et al. Hepatocellular Carcinomas With Mutational Activation of Beta-Catenin Require Choline and Can Be Detected by Positron Emission Tomography. Gastroenterology 2019;
20. Benhamouche, S, Decaens, T, Godard, C, Chambrey, R, Rickman, D S, Moinard, C et al. Apc tumor suppressor gene is the “zonation-keeper” of mouse liver. Developmental cell 2006; 10:759-770
21. Loesch, R, Caruso, S, Paradis, V, Godard, C, Gougelet, A, Renault, G et al. Deleting the beta-catenin degradation domain in mouse hepatocytes drives hepatocellular carcinoma or hepatoblastoma-like tumor growth. J Hepatol 2022;
22. Hooks, K B, Audoux, J, Fazli, H, Lesjean, S, Ernault, T, Dugot-Senant, N et al. New insights into diagnosis and therapeutic options for proliferative hepatoblastoma. Hepatology 2018; 68:89-102
23. Hu, S, Cao, C, Poddar, M, Delgado, E, Singh, S, Singh-Varma, A et al. Hepatocyte beta-catenin loss is compensated by Insulin-mTORC1 activation to promote liver regeneration. Hepatology 2022;
24. Yin, C, Wang, P Q, Xu, W P, Yang, Y, Zhang, Q, Ning, B F et al. Hepatocyte nuclear factor-4alpha reverses malignancy of hepatocellular carcinoma through regulating miR-134 in the DLK1-DIO3 region. Hepatology 2013; 58:1964-1976
25. Braem, C, Recolin, B, Rancourt, R C, Angiolini, C, Barthes, P, Branchu, P et al. Genomic matrix attachment region and chromosome conformation capture quantitative real time PCR assays identify novel putative regulatory elements at the imprinted Dlk1/Gt12 locus. J Biol Chem 2008; 283:18612-18620
26. Lleres, D, Moindrot, B, Pathak, R, Piras, V, Matelot, M, Pignard, B et al. CTCF modulates allele-specific sub-TAD organization and imprinted gene activity at the mouse Dlk1-Dio3 and Igf2-H₁₉ domains. Genome Biol 2019; 20:272
27. Ran, F A, Cong, L, Yan, W X, Scott, D A, Gootenberg, J S, Kriz, A J et al. In vivo genome editing using Staphylococcus aureus Cas9. Nature 2015; 520:186-191
28. Colnot, S, Decaens, T, Niwa-Kawakita, M, Godard, C, Hamard, G, Kahn, A et al. Liver-targeted disruption of Apc in mice activates beta-catenin signaling and leads to hepatocellular carcinomas. Proc Natl Acad Sci USA 2004; 101:17216-17221
29. Wierstra, I. FOXMI (Forkhead box M1) in tumorigenesis: overexpression in human cancer, implication in tumorigenesis, oncogenic functions, tumor-suppressive properties, and target of anticancer therapy. Adv Cancer Res 2013; 119:191-419
30. Hirsch, T Z, Negulescu, A, Gupta, B, Caruso, S, Noblet, B, Couchy, G et al. BAP1 mutations define a homogeneous subgroup of hepatocellular carcinoma with fibrolamellar-like features and activated PKA. J Hepatol 2020; 72:924-936
31. Bayard, Q, Meunier, L, Peneau, C, Renault, V, Shinde, J, Nault, J C et al. Cyclin A2/E1 activation defines a hepatocellular carcinoma subclass with a rearrangement signature of replication stress. Nat Commun 2018; 9:5235
32. Labialle, S, Marty, V, Bortolin-Cavaille, M L, Hoareau-Osman, M, Pradere, J P, Valet, P et al. The miR-379/miR-410 cluster at the imprinted Dlk1-Dio3 domain controls neonatal metabolic adaptation. EMBO J 2014; 33:2216-2230
33. Anwar, S L, Krech, T, Hasemeier, B, Schipper, E, Schweitzer, N, Vogel, A et al. Loss of imprinting and allelic switching at the DLK1-MEG3 locus in human hepatocellular carcinoma. PloS one 2012; 7: e49462
34. Riordan, J D, Keng, V W, Tschida, B R, Scheetz, T E, Bell, J B, Podetz-Pedersen, K M et al. Identification of rtl1, a retrotransposon-derived imprinted gene, as a novel driver of hepatocarcinogenesis. PLOS genetics 2013; 9: e1003441
35. Donsante, A, Miller, D G, Li, Y, Vogler, C, Brunt, E M, Russell, D W et al. AAV vector integration sites in mouse hepatocellular carcinoma. Science 2007; 317:477
36. Ranzani, M, Cesana, D, Bartholomae, C C, Sanvito, F, Pala, M, Benedicenti, F et al.

Lentiviral vector-based insertional mutagenesis identifies genes associated with liver cancer. Nature methods 2013; 10:155-161
37. Zhang, L, Yang, Z, Trottier, J, Barbier, O & Wang, L. Long noncoding RNA MEG3 induces cholestatic liver injury by interaction with PTBP1 to facilitate shp mRNA decay. Hepatology 2017; 65:604-615
38. Zhu, X, Wu, Y B, Zhou, J & Kang, D M. Upregulation of lncRNA MEG3 promotes hepatic insulin resistance via increasing FoxO1 expression. Biochem Biophys Res Commun 2016; 469:319-325
39. Dai, R, Lu, R & Ahmed, S A. The Upregulation of Genomic Imprinted DLK1-Dio3 miRNAs in Murine Lupus Is Associated with Global DNA Hypomethylation. PLOS One 2016; 11: e0153509
40. Dai, R, Zhang, Y, Khan, D, Heid, B, Caudell, D, Crasta, O et al. Identification of a common lupus disease-associated microRNA expression pattern in three different murine models of lupus. PLOS One 2010; 5: e14302
41. Essandoh, K, Li, Y, Huo, J & Fan, G C. MiRNA-Mediated Macrophage Polarization and its Potential Role in the Regulation of Inflammatory Response. Shock 2016; 46:122-131
42. Ying, H, Kang, Y, Zhang, H, Zhao, D, Xia, J, Lu, Z et al. MiR-127 modulates macrophage polarization and promotes lung inflammation and injury by activating the JNK pathway. J Immunol 2015; 194:1239-1251
43. Yochum, G S, Cleland, R & Goodman, R H. A genome-wide screen for beta-catenin binding sites identifies a downstream enhancer element that controls c-Myc gene expression. Mol Cell Biol 2008; 28:7368-7379
44. Yochum, G S, Sherrick, C M, Macpartlin, M & Goodman, R H. A beta-catenin/TCF-coordinated chromatin loop at MYC integrates 5′ and 3′ Wnt responsive enhancers. Proc Natl

Acad Sci USA 2010; 107: 145-150
45. Korostowski, L, Raval, A, Breuer, G & Engel, N. Enhancer-driven chromatin interactions during development promote escape from silencing by a long non-coding RNA. Epigenetics Chromatin 2011; 4:21
46. Eun, B, Sampley, M L, Good, A L, Gebert, C M & Pfeifer, K. Promoter cross-talk via a shared enhancer explains paternally biased expression of Nctc1 at the Igf2/H₁₉/Nctc1 imprinted locus. Nucleic Acids Res 2013; 41:817-826
47. Kota, S K, Lleres, D, Bouschet, T, Hirasawa, R, Marchand, A, Begon-Pescia, C et al. ICR noncoding RNA expression controls imprinting and DNA replication at the Dlk1-Dio3 domain. Developmental cell 2014; 31:19-33
48. Chen, T, Tian, L, Chen, J, Zhao, X, Zhou, J, Guo, T et al. A Comprehensive Genomic Analysis Constructs miRNA-mRNA Interaction Network in Hepatoblastoma. Front Cell Dev Biol 2021; 9:655703
49. Hadjihannas, M V, Bernkopf, D B, Bruckner, M & Behrens, J. Cell cycle control of Wnt/beta-catenin signalling by conductin/axin2 through CDC20. EMBO Rep 2012; 13:347-354
50. Love, M I, Huber, W & Anders, S. Moderated estimation of fold change and dispersion for 5 RNA-seq data with DESeq2. Genome Biol 2014; 15:550
51. Boyault, S, Rickman, D S, de Reynies, A, Balabaud, C, Rebouissou, S, Jeannot, E et al. Transcriptome classification of HCC is related to gene alterations and to new therapeutic targets. Hepatology 2007; 45:42-52

1. A method of treating a liver cancer in a patient harbouring at least one activating mutation in CTNNB1 gene comprising administering to the patient an agent capable of disrupting a b-catenin/TCF-4 binding site located upstream of Meg3 in a DLK1/DIO3 locus.

2. The method of claim 1 wherein the agent is a DNA-targeting endonuclease that cleaves genomic DNA of cancer cells in at least one position of the b-catenin/TCF-4 binding site, thereby repressing transcription of the DLK1/DIO3 locus.

3. The method of claim 2, wherein the cleavage of the genomic DNA leads to genome editing of the b-catenin/TCF-4 binding site located upstream of Meg3 and prevents binding of a transcriptional b-catenin/TCF-4 complex to its binding site.

4. The method of claim 1, wherein the patient suffers from a hepatocellular carcinoma or from a hepatoblastoma.

5. The method of claim 2, wherein the DNA targeting endonuclease is a TALEN.

6. The method of claim 2, wherein the DNA targeting endonuclease is a ZFN.

7. The method of claim 2, wherein the DNA targeting endonuclease is a CRISPR-associated endonuclease.

8. The method of claim 7 wherein the CRISPR-associated endonuclease is a Cas9 nuclease.

9. The method of claim 7 further comprising administering an effective amount of one or more guide RNA(s) with the CRISPR-associated endonuclease.

10. The method of claim 9 wherein the one or more guide RNAs recruits the CRISPR-associated endonuclease to the b-catenin/TCF-4 binding site located upstream of Meg3 thereby generating DNA double-strand breaks (DSBs).

11. The method of claim 9 wherein a combination of a first and a second guide RNA is administered and wherein the first guide RNA targets the sequence SEQ ID NO:4 and the second guide RNA targets at least one sequence selected from the group consisting of SEQ ID NO:3, SEQ ID NO:5 and SEQ ID NO:6.

12. The method of claim 9 wherein a combination of a first and a second guide RNA is administered and wherein the first guide RNA is encoded by SEQ ID NO:8 and the second guide RNA is encoded by a sequence selected from the group consisting of SEQ ID NO:7, SEQ ID NO:9, and SEQ ID NO: 10.

13. The method of claim 9 wherein the CRISPR-associated endonuclease and the one or more guide RNAs are expressed from one or more vectors.

14. The method of claim 13 wherein the CRISPR endonuclease and the one or more guide RNAs are encoded by the same nucleic acid.

15. The method of claim 13 wherein the one or more vectors is a viral vector.

16. The method of claim 15 wherein the viral vector is an adeno-associated virus (AAV) vector.

17. The method of claim 16, wherein the AAV vector is an AAV8 vector.

18. The method of claim 17, wherein the AAV8 vector is an AAV2/AAV8 vector.


SEQUENCE LISTING (43 sequences)

SEQ ID NO: 1 - skipped

SEQ ID NO: 2 - DNA, 828 bp, Homo sapiens
  source 1..828: mol_type = genomic DNA
attgcttgagcccaggagtttgaggctgcaatgaaccatgattgcaccactgtactccagcctgggcaacaaagtgagatctcgtcccaaaaaaagataaataaaaagaaaatagtctgttttagtctgtattcagagcaagcctgtggcatgaatatcaactttcctgtttttgcaggggggaaaccgaggcctggcagggcgaagtgggcaggacccttctcaaagggccagggtgctagtggccactgtccttcccccaccacctccacgcccccatccccccacagttcacgactgcaaaggcagctttaggttgggagctggtggagcaaaaaggccgttcaaaggcagccttcgtttgcttttccttatcatcacatgtgcaagggcagctccgtttacctagaggcaggcgtctttgagctcatcctttccgcaaacacctaaagggtatgttgaacatttgagcccctgtctcagagggcgcagcgtttgcacaacataagccctggcggctcggtagacgggtatggggggtagactcaggtttctggatgaagcactgagagccaaggtgacctgcggtgttccgggatgacctttggtgaccttcggtcctgtccagtgacctggggtggtgaagttaccccaaatgggaggaaatgaactctgggtgggcgaaggctgcaggacgtgttggcatgatggctgtagaaagtgcacaccagggctggtgtggagggcgcctcggctgctctgagaatcttccactaccccggcctgggattcccaggaaaggctggtggtggaggccagagaggctca

SEQ ID NO: 3 - DNA, 21 bp, Homo sapiens
  source 1..21: mol_type = genomic DNA
gcaggacccttctcaaagggc

SEQ ID NO: 4 - DNA, 21 bp, Homo sapiens
  source 1..21: mol_type = genomic DNA
tccgcaaacacctaaagggta

SEQ ID NO: 5 - DNA, 21 bp, Homo sapiens
  source 1..21: mol_type = genomic DNA
ccacagttcacgactgcaaag

SEQ ID NO: 6 - DNA, 21 bp, Homo sapiens
  source 1..21: mol_type = genomic DNA
tattcagagcaagcctgtggc

SEQ ID NO: 7 - DNA, 27 bp, synthetic construct
  misc_feature 1..27: note = sg3 encoding sequence
  source 1..27: mol_type = other DNA
gcaggacccttctcaaagggccagggt

SEQ ID NO: 8 - DNA, 27 bp, synthetic construct
  misc_feature 1..27: note = sg2Bis encoding sequence
  source 1..27: mol_type = other DNA
taccctttaggtgtttgcggaaaggat

SEQ ID NO: 9 - DNA, 27 bp, synthetic construct
  misc_feature 1..27: note = sg5 encoding sequence
  source 1..27: mol_type = other DNA
ctttgcagtcgtgaactgtggggggat

SEQ ID NO: 10 - DNA, 27 bp, synthetic construct
  misc_feature 1..27: note = sg1 encoding sequence
  source 1..27: mol_type = other DNA
tattcagagcaagcctgtggcatgaat

SEQ ID NO: 11 - DNA, 27 bp, synthetic construct
  misc_feature 1..27: note = murine sg2 encoding sequence
  source 1..27: mol_type = other DNA
ttcctcagtggggctaaaggagagggt

SEQ ID NO: 12 - DNA, 27 bp, synthetic construct
  misc_feature 1..27: note = murine sg5 encoding sequence
  source 1..27: mol_type = other DNA
ggatgacctttgacttctgaagggagt

SEQ ID NO: 13 - DNA, 27 bp, synthetic construct
  misc_feature 1..27: note = sgRNA against Rosa26 locus
  source 1..27: mol_type = other DNA
ctcgatggaaaatactccgaggcggat

SEQ ID NO: 14 - DNA, 20 bp, synthetic construct
  source 1..20: mol_type = other DNA
agcatggccgagtactcatt

SEQ ID NO: 15 - DNA, 20 bp, synthetic construct
  source 1..20: mol_type = other DNA
cctctgcatgacctgtgact

SEQ ID NO: 16 - DNA, 20 bp, synthetic construct
  source 1..20: mol_type = other DNA
cttgctctcccaaagtcgct

SEQ ID NO: 17 - DNA, 20 bp, synthetic construct
  source 1..20: mol_type = other DNA
ccaatgctctgtctaggggt

SEQ ID NO: 18 - DNA, 20 bp, synthetic construct
  source 1..20: mol_type = other DNA
tgaggctgcaatgaaccatg

SEQ ID NO: 19 - DNA, 20 bp, synthetic construct
  source 1..20: mol_type = other DNA
ggcttatgttgtgcaaacgc

SEQ ID NO: 20 - DNA, 20 bp, synthetic construct
  source 1..20: mol_type = other DNA
gtaacccgttgaaccccatt

SEQ ID NO: 21 - DNA, 20 bp, synthetic construct
  source 1..20: mol_type = other DNA
ccatccaatcggtagtagcg

SEQ ID NO: 22 - DNA, 20 bp, synthetic construct
  source 1..20: mol_type = other DNA
gccatctacctctgagtccc

SEQ ID NO: 23 - DNA, 20 bp, synthetic construct
  source 1..20: mol_type = other DNA
agagcagaaacccctccttc

SEQ ID NO: 24 - DNA, 20 bp, synthetic construct
  source 1..20: mol_type = other DNA
ccaggttcaaggtccctcat

SEQ ID NO: 25 - DNA, 20 bp, synthetic construct
  source 1..20: mol_type = other DNA
tcttgtgtctcgaaggcctt

SEQ ID NO: 26 - DNA, 20 bp, synthetic construct
  source 1..20: mol_type = other DNA
ctgcctgcgaagagagcatc

SEQ ID NO: 27 - DNA, 20 bp, synthetic construct
  source 1..20: mol_type = other DNA
agctccacttcgccttttgg

SEQ ID NO: 28 - DNA, 22 bp, synthetic construct
  source 1..22: mol_type = other DNA
aagtggtgagcggctaaaggag

SEQ ID NO: 29 - DNA, 22 bp, synthetic construct
  source 1..22: mol_type = other DNA
gaagccttggaacacacgagtc

SEQ ID NO: 30 - DNA, 22 bp, synthetic construct
  source 1..22: mol_type = other DNA
ggatgacctagacgtgcttacc

SEQ ID NO: 31 - DNA, 22 bp, synthetic construct
  source 1..22: mol_type = other DNA
tcgcttgagtggtaaggacagc

SEQ ID NO: 32 - DNA, 23 bp, synthetic construct
  source 1..23: mol_type = other DNA
cctctatggtcagaatgcagcag

SEQ ID NO: 33 - DNA, 22 bp, synthetic construct
  source 1..22: mol_type = other DNA
actgcttgaactcctctcgctc

SEQ ID NO: 34 - DNA, 22 bp, synthetic construct
  source 1..22: mol_type = other DNA
ttcctccaagaggaaggctctc

SEQ ID NO: 35 - DNA, 22 bp, synthetic construct
  source 1..22: mol_type = other DNA
ggtgtcttggtcagtgagcact

SEQ ID NO: 36 - DNA, 22 bp, synthetic construct
  source 1..22: mol_type = other DNA
caagcgagaagtgaaggttgcc

SEQ ID NO: 37 - DNA, 22 bp, synthetic construct
  source 1..22: mol_type = other DNA
gctacccacaaaattctgcgcc

SEQ ID NO: 38 - DNA, 22 bp, synthetic construct
  source 1..22: mol_type = other DNA
gcacgacttcagttacaggagc

SEQ ID NO: 39 - DNA, 24 bp, synthetic construct
  source 1..24: mol_type = other DNA
tgctttggtgttttctctgtagtc

SEQ ID NO: 40 - DNA, 22 bp, synthetic construct
  source 1..22: mol_type = other DNA
tactgaccagcgcagatacacg

SEQ ID NO: 41 - DNA, 22 bp, synthetic construct
  source 1..22: mol_type = other DNA
tcctttgccagttctccactcc

SEQ ID NO: 42 - DNA, 21 bp, synthetic construct
  source 1..21: mol_type = other DNA
gccttcaccattcatgtggat

SEQ ID NO: 43 - DNA, 21 bp, synthetic construct
  source 1..21: mol_type = other DNA
ttgctccgggtaaagagacag